(19) United States
(12) Patent Application Publication
(10) Pub. No.: US 2024/0115732 A1
(43) Pub. Date: Apr. 11, 2024

(54) Epicardial Delivery of Gene Therapy

(71) Applicant: XyloCor Therapeutics, Inc., Wayne, PA (US)

(72) Inventors: Rickey Reinhardt, Brielle, NJ (US); Todd Rosengart, Houston, TX (US); Ronald Crystal, New York, NY (US)

(21) Appl. No.: 17/767,524

(22) PCT Filed: Oct. 9, 2020
(86) PCT No.: PCT/US2020/055082

§ 371 Date: Apr. 8, 2022

Related U.S. Application Data
(60) Provisional application No. 62/912,958, filed on Oct. 9, 2019.

(51) Int. Cl. A61K 48/00; C07K 14/475; C12N 15/86

(52) U.S. Cl. CPC A61K 48/005; C07K 14/475; C12N 15/86; A61K 38/00; C12N 2710/10343

(57) ABSTRACT

In various aspects and embodiments, the invention provides a method of treating a cardiovascular disease in a subject in need thereof, the method comprising administering an effective amount of a viral vector comprising a therapeutic polynucleotide directly into the heart of the subject. In various embodiments, the pharmaceutical composition is administered through a series of 15 injections at separate delivery sites in the heart of the subject, and wherein the viral vector diffuses through substantially all of the heart.

### CROSS REFERENCE TO RELATED APPLICATIONS

The present application claims priority under 35 U.S.C. § 119(e) to U.S. Provisional Patent Application No. 62/912,958 filed Oct. 9, 2019, which is incorporated herein by reference in its entirety.

### BACKGROUND OF THE INVENTION

According to the American Heart Association 2019 Heart Disease and Stroke Statistics Update, the prevalence of cardiovascular disease in US adults≥20 years of age is 48.0% overall (121.5 million in 2016) and cardiovascular disease accounts for nearly 841,000 deaths in the US (≈17.6 million globally). An estimated 18.2 million Americans ≥20 years of age have coronary heart disease and it is the leading cause (43.2%) of deaths attributable to cardiovascular disease in the US. From 2006 to 2016, the annual death rate attributable to coronary heart disease declined 31.8% but the burden and risk factors remain alarmingly high. Ischemic heart disease is characterized by reduced blood flow to the heart. Chronic ischemia is caused by narrowing of the coronary arteries, which limits blood supply to areas of the muscle. Acute ischemia results from a sudden plaque that ruptures. If left untreated, coronary artery disease (CAD) progresses to worsening symptoms and morbidity and can result in myocardial infarction (MI) or death. It has been reported that as the population ages and as the incidence of obesity and diabetes approaches epidemic proportions, the number of patients with severe CAD will continue to grow.

In spite of evidence that rates of mortality in refractory angina may be decreasing with newer therapies, the number of patients experiencing disabling angina that is not amenable to surgical or percutaneous coronary revascularization despite optimal available medical therapy (“no option patients”) is anticipated to rise. According to data from NHANES 2013 to 2016, the overall prevalence for angina is 3.6% in US adults≥20 years of age (9.4 million). Among patients with a history of CAD (acute coronary syndrome, prior coronary revascularization procedure or stable angina), 32.7% self-reported at least 1 episode of angina over the past month. Of those patients reporting angina, 23.3% reported daily or weekly symptoms of angina, and 56.3% of these patients with daily or weekly angina were taking at least 2 antianginal medications. Estimates suggest that in the US between 600,000 and 1.8 million patients suffer from refractory angina, with 75,000 to 200,000 new patients diagnosed each year, while in Europe, 30,000-50,000 new cases are diagnosed per year.

Treatment options for CAD and refractory angina include medical therapy, balloon angioplasty (with or without stenting), atherectomy and bypass surgery. Pharmacologic therapy is a mainstay of disease management for most forms of CAD and specifically for refractory angina. Pharmacological treatment includes first-line therapy with beta-blockers, calcium channel blockers, nitrates, second-line therapy with ranolazine and additional therapies to reduce the risk of MI and/or death including antiplatelet therapy, lipid-lowering therapy, and angiotensin-converting enzyme (ACE) inhibitors. At best, pharmacological therapy treats the symptoms and prevents further disease progression but does not reverse the pathology of the disease. When the condition cannot be effectively treated with medicines or catheter-based angioplasty and stents, CABG may be recommended. CABG uses arteries and/or veins from other parts of the body to bypass the blocked coronary arteries on the surface of the heart. Despite the expense and the procedure-related patient morbidity and mortality, these procedures do not provide long-term relief of symptoms, and oftentimes repeat surgical intervention is required. Anatomic reasons which preclude current revascularization procedures include severe diffuse CAD, collateral-dependent myocardium, multiple coronary restenosis, chronic total coronary occlusions, degenerated saphenous vein grafts, poor distal targets or lack of conduits due to prior CABG in addition to a number of comorbidities. The growing incidence of diabetes predisposes individuals to this anatomic substrate. If pharmaceutical options have also been exhausted, only life-style alterations remain, and severe restrictions may result. With improvement in therapies, a growing number of patients with CAD survive to a point where conventional therapeutic options have been exhausted. Medical and surgical treatments can often provide adequate short-term treatment for individuals with CAD, but there is still a major need for improvement over the current modalities, specifically for those individuals who are unsuitable for PCI or CABG, in whom bypass surgery is applicable to only limited regions of the myocardium, and in whom medical therapy is unsuccessful. The relationship between myocardial ischemia, cardiac pain and unsuitability for revascularization has led to a need for new therapies. This disclosure addresses that need.

### SUMMARY OF THE INVENTION

In one aspect, the invention provides a method of treating a cardiovascular disease in a subject in need thereof, the method comprising administering directly into the heart of the subject during Transthoracic Epicardial Procedure (TECAP) an effective amount of pharmaceutical composition comprising a viral vector comprising a therapeutic polynucleotide.

In various embodiments, the pharmaceutical composition is administered through a series of 15 injections at separate delivery sites in the heart of the subject, and wherein the viral vector diffuses through substantially all of the heart.

In various embodiments, the viral vector is an adenoviral vector.

In various embodiments, the viral vector comprises a polynucleotide encoding one or more isoforms of VEGF.

In various embodiments, the heart of the subject is visualized throughout the procedure using a thorascope.

In various embodiments, a dose of the viral vector of about 1×10⁹ vp, about 1×10¹⁰ vp, about 4×10¹⁰ vp or about 1×10¹¹ vp is administered.

In various embodiments, each injection has an injection volume of about 0.1 mL.

In various embodiments, the cardiovascular disease is coronary artery disease.

In various embodiments, the TECAP comprises making a 4-5 cm anterolateral incision in the 5th to 7th intercostal space of the subject.

In various embodiments, the injections are made in the left ventricle.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings

FIG. 1 depicts a sample injection grid indicating spacing between injection sites in one embodiment of the invention.

FIG. 2 depicts a schematic diagram of the study design for the study described in Example 1.

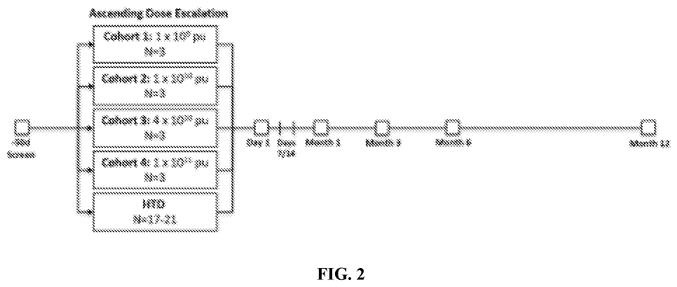

FIG. 3 depicts the genetic structure of AdVEGF-All6A+.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “coronary artery disease or “CAD” means the narrowing or blockage of coronary arteries usually caused by atherosclerosis or the buildup of plaque on the inner walls of the arteries in the heart which can restrict blood flow to areas of the heart.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, intramuscular, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “TECAP” or “Transthoracic Epicardial Procedure”, refers to a minimally invasive surgical approach for transthoracic epicardial access.

As used herein, “treating a disease or disorder” means reducing the frequency with which a symptom of the disease or disorder is experienced by a patient or improving patient ability to function. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

As used herein, the term “VEGF” refers to the gene or protein vascular epithelial growth factor. A person of skill in the art is familiar with VEGF and its isoforms. See, e.g., Yla-Herttuala et al Vascular Endothelial Growth Factors Biology and Current Status of Clinical Applications in Cardiovascular Medicine. J Am Coll Cardiol 2007; 49:1015-26.

As used here in the terms “vp” or “viral particles” means as total number of functional (infectiouse) and non-functional (non-infectiouse) virus particles.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

In one aspect, the invention provides a method of treating a cardiovascular disease in a subject in need thereof, the method comprising administering directly into the heart of the subject a pharmaceutical composition comprising an effective amount of a viral vector comprising a therapeutic polynucleotide. Effective administration of VEGF to patients in need of treatment for cardiovascular disease has proven elusive. It has now been surprisingly discovered that direct injection of a viral vector comprising a therapeutic polynucleotide to the heart of a subject, thereby expresses the corresponding polypeptide in the heart of the subject, is a safe and effective method of treating cardiovascular disease. In various embodiments, the cardiovascular disease may be any cardiovascular disease that may be treated by inducing angiogenesis in the subject's heart. In various embodiments, the cardiovascular disease is coronary artery disease. Without meaning to be limited by theory, it is presently believed that direct injection to the heart, in various embodiments, allows lower doses of viral vector to provide greater benefit to the treatment of the subject and thereby avoids various disadvantages of systemic administration.

### Viral Vector Comprising One or More Isoforms of VEGF

In various embodiments the therapeutic agent delivered by the methods taught herein is a viral vector comprising a polynucleotide encoding one or more isoforms of VEGF and configured to express polypeptides corresponding to the one or more isoforms of VEGF in a tissue of the subject. In various embodiments, the viral vector is an adenoviral vector. A person of skill in the art is familiar with various viral vectors that are suitable for the practice of the various embodiments of the invention. In various embodiments, the viral vector is deficient in various genes that allow the wild type vector to replicate and therefore is suitable for gene transfer to a subject. In various embodiments, the therapeutic polynucleotide encodes one or more of VEGF isoforms 121, 165 and 189. In various embodiments, the therapeutic polynucleotide encodes VEGF isoforms 121, 165 and 189. In various embodiments the viral vector is as described in U.S. Pat. Nos. 6,518,255 and 7,368,553, each of which is hereby incorporated by reference in their entirety.

In various embodiments, the therapeutic agent is an adenoviral vector in formulation buffer in a suitable container closure system, such as vials, pre-filled syringes or other container. The drug product can be in different presentations, such as lyophilized (freeze-dried) form or other physical state. The drug product can be composed of any combination of a drug and a device (a combination drug product) including robotic, semi-robotic or nonrobotic devices.

In various embodiments, the viral vector is AdVEGFXC1, a replication-deficient, recombinant human adenovirus serotype 5 (Ad5) viral vector with an expression cassette for human vascular endothelial growth factor (VEGF) that includes introns and splice sites to generate multiple naturally occurring isoforms of VEGF, including VEGF121, VEGF165 and VEGF189. In various embodiments, the VEGF expression cassette is inserted in the E1 region of the adenovirus backbone, which has deleted the adenovirus E1A and E3 genes, and partially deleted E1B. The full DNA sequence analysis of AdVEGFAll6A+ is provided in SEQ ID NO: 1, and has been confirmed by Annotation for transgene region of the virus genome—provided in Table 1.

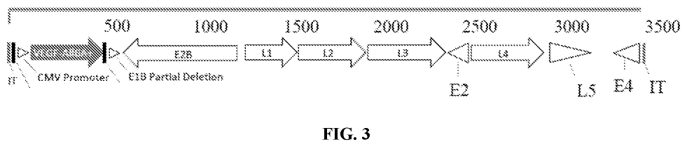

The genetic structure of AdVEGF-All6A+ is shown in FIG. 3. The position of the human VEGF cDNA/genomic hybrid expression cassette is indicated by the grey arrow. Positions of the Ad early genes (E2A, E2B and E4), Ad late genes (L1, L2, L3, L4 and L5), inverted terminal repeats (ITR) and encapsidation signal (ES) are also indicated.

TABLE 1
Features Annotation for AdVEGFAll6A+

| Feature | Location (bp) |
|---|---|
| Ad5 ITR | 1-103 |
| Ad5 ψ | 191-341 |
| VEGF expression cassette | 399-5590 |
| CMV promoter | 399-906 |
| VEGF exons 1-5 (partial cDNA) | 1060-1481 |
| VEGF exon 6 (splice modified) | 3295-3366 |
| VEGF exon 7 | 4519-4650 |
| VEGF exon 8 | 5298-5316 |
| SV40 polyA | 5469-5590 |
| Ad5 backbone (E1 deleted) | 5600-35371 |
| Ad5 ITR | 35269-35371 |

| DNA sequence of AdVEGFAll6A+ | |
|---|---|
| SEQ ID NO: 1 | |
| CATCATCAAT AATATACCTT ATTTTGGATT GAAGCCAATA TGATAATGAG | 50 |
| GGGGTGGAGT TTGTGACGTG GCGCGGGGCG TGGGAACGGG GCGGGTGACG | 100 |
| TAGTAGTGTG GCGGAAGTGT GATGTTGCAA GTGTGGCGGA ACACATGTAA | 150 |
| GCGACGGATG TGGCAAAAGT GACGTTTTTG GTGTGCGCCG GTGTACACAG | 200 |
| GAAGTGACAA TTTTCGCGCG GTTTTAGGCG GATGTTGTAG TAAATTTGGG | 250 |
| CGTAACCGAG TAAGATTTGG CCATTTTCGC GGGAAAACTG AATAAGAGGA | 300 |
| AGTGAAATCT GAATAATTTT GTGTTACTCA TAGCGCGTAA TACTGTAATA | 350 |
| GTAATCAATT ACGGGGTCAT TAGTTCATAG CCCATATATG GAGTTCCGCG | 400 |
| TTACATAACT TACGGTAAAT GGCCCGCCTG GCTGACCGCC CAACGACCCC | 450 |
| CGCCCATTGA CGTCAATAAT GACGTATGTT CCCATAGTAA CGCCAATAGG | 500 |
| GACTTTCCAT TGACGTCAAT GGGTGGAGTA TTTACGGTAA ACTGCCCACT | 550 |
| TGGCAGTACA TCAAGTGTAT CATATGCCAA GTACGCCCCC TATTGACGTC | 600 |
| AATGACGGTA AATGGCCCGC CTGGCATTAT GCCCAGTACA TGACCTTATG | 650 |
| GGACTTTCCT ACTTGGCAGT ACATCTACGT ATTAGTCATC GCTATTACCA | 700 |
| TGGTGATGCG GTTTTGGCAG TACATCAATG GGCGTGGATA GCGGTTTGAC | 750 |
| TCACGGGGAT TTCCAAGTCT CCACCCCATT GACGTCAATG GGAGTTTGTT | 800 |
| TTGGCACCAA AATCAACGGG ACTTTCCAAA ATGTCGTAAC AACTCCGCCC | 850 |
| CATTGACGCA AATGGGCGGT AGGCGTGTAC GGTGGGAGGT CTATATAAGC | 900 |
| AGAGCTGGTT TAGTGAACCG TCAGATCCGC TAGAGATCTG GTACCGGGCC | 950 |
| CCCCCTCGAG GTCGACGGTA TCGATAAGCT TGATATCGAA TTCCTGCAGT | 1000 |
| CACCGTCGTC GACGGTATCG ATAAGCTTGA TATCGAATTC CGGTCGGGCC | 1050 |
| TCCGAAACCA TGAACTTTCT GCTGTCTTGG GTGCATTGGA GCCTTGCCTT | 1100 |
| GCTGCTCTAC CTCCACCATG CCAAGTGGTC CCAGGCTGCA CCCATGGCAG | 1150 |
| AAGGAGGAGG GCAGAATCAT CACGAAGTGG TGAAGTTCAT GGATGTCTAT | 1200 |
| CAGCGCAGCT ACTGCCATCC GATCGAGACC CTGGTGGACA TCTTCCAGGA | 1250 |
| GTACCCTGAT GAGATCGAGT ACATCTTCAA GCCATCCTGT GTGCCCCTGA | 1300 |
| TGCGATGCGG GGGCTGCTGC AATGACGAGG GCCTGGAGTG TGTGCCCACT | 1350 |
| GAGGAGTCCA ACATCACCAT GCAGATTATG CGGATCAAAC CTCACCAAGG | 1400 |
| CCAGCACATA GGAGAGATGA GCTTCCTACA GCACAACAAA TGTGAATGCA | 1450 |
| GACCAAAGAA AGATAGAGCT CGACAAGAAA AGTAAGTGGC CCTGACTTTA | 1500 |
| GCACTTCTCC CTCTCCATGG CCGGTTGTCT TGGTTTGGGG CTCTTGGCTA | 1550 |
| CCTCTGTTGG GGGCTCCCAT AGCCTCCCTG GGTCAGGGAC TTGGTCTTGT | 1600 |
| GGGGGACTTG TGGTGGCAGC AACAATGGGA TGGAGCCAAC TCCAGGATGA | 1650 |
| TGGCTCTAGG GCTAGTGAGA AAACATAGCC AGGAGCCTGG CACTTCCTTT | 1700 |
| GGAAGGGACA ATGCCTTCTG GGTCTCCAGA TCATTTCTGA CCAGGACTTG | 1750 |
| CTGTTTCGGT GTGTCAGGGG GCACTGTGGA CACTGGCTCA CTGGCTTGCT | 1800 |
| CTAGGACACC CACAGTGGGG AGAGGGAGTG GGTGGCAGAG AGGCCAGCTT | 1850 |
| TTGTGTGTCA GAGGAAATGG CCTCTTTTGG TGGCTGCTGT GACGGTGCAG | 1900 |
| TTGGATGCGA GGCCGGCTGG AGGGTGGTTT CTCAGTGCAT GCCCTCCTGT | 1950 |
| AGGCGGCAGG CGGCAGACAC ACAGCCCTCT TGGCCAGGGA GAAAAAGTTG | 2000 |
| AATGTTGGTC ATTTTCAGAG GCTTGTGAGT GCTCCGTGTT AAGGGGCAGG | 2050 |
| TAGGATGGGG TGGGGGACAA GGTTTGGCGG CAGTAACCCT TCAAGACAGG | 2100 |
| GTGGGCGGCT GGCATCAGCA AGAGCTTGCA GGGAAAGAGA GACTGAGAGA | 2150 |
| GAGCACCTGT GCCCTGCCCT TTCCCCCACA CCATCTTGTC TGCCTCCAGT | 2200 |
| GCTGTGCGGA CATTGAAGCC CCCACCAGGC CTCAACCCCT TGCCTCTTCC | 2250 |
| CTCAGCTCCC AGCTTCCAGA GCGAGGGGAT GCGGAAACCT TCCTTCCACC | 2300 |
| CTTTGGTGCT TTCTCCTAAG GGGGACAGAC TTGCCCTCTC TGGTCCCTTC | 2350 |
| TCCCCCTCCT TTCTTCCCTG TGACAGACAT CCTGAGGTGT GTTCTCTTGG | 2400 |
| GCTTGGCAGG CATGGAGAGC TCTGGTTCTC TTGAAGGGGA CAGGCTACAG | 2450 |
| CCTGCCCCCC TTCCTGTTTC CCCAAATGAC TGCTCTGCCA TGGGGAGAGT | 2500 |
| AGGGGGCTCG CCTGGGCTCG GAAGAGTGTC TGGTGAGATG GTGTAGCAGG | 2550 |
| CTTTGACAGG CTGGGGAGAG AACTCCCTGC CAAGTACCGC CCAAGCCTCT | 2600 |
| CCTCCCCAGA CCTCCTTAAC TCCCACCCCA TCCTGCTGCC TGCCCAGGGC | 2650 |
| TCCAGGACAC CCAGCCCTGC CTCCCAGTCC AGGTCGTGCT GAGCAGGCTG | 2700 |
| GTGTTGCTCT TGGTTCCGTG CCAGCTCCCA AGGTAGCCGC TTCCCCCACA | 2750 |
| CCGGGATTCC CAGAGGTTCT GTCGCAGTTG CAAATGAAGG CACAAGGCCT | 2800 |
| GATACACAGC CCTCCCTCCC ACTCCTGCTC CCCATCCAGG CAGGTCTCTG | 2850 |
| ACCTTCTCCC CAAAGTCTGG CCTACCTTTT ATCACCCCCG GACCTTCAGG | 2900 |
| GTCAGACTTG GACAGGGCTG CTGGGCAAAG AGCCTTCCCT CAGGCTTTGC | 2950 |
| CCCCTGCCGG GGACTGGGAG CCACTGTGAG TGTGGAGACC TTTGGGTCCT | 3000 |
| GTGCCCTCCA CCCAGTCTCG GCTTCCCACC AAAGCCTTGT CAGGGGCTGG | 3050 |
| GTTTGCCATC CCATGGTGGG CAGCGTGAGG AGAAGAAAGA GCCATCGAGT | 3100 |
| GCTTGCTGCC CAGACACGCC TGTGTGCGCC CGCGCATGCC TCCCCAGAGA | 3150 |
| CCACCTGCCT CCTGACACTT CCTCCGGGAA GCGGCCCTGT GTGGCTTTGC | 3200 |
| TTTGGTCGTT CCCCCATCCC TGCCCCCCTG AGCACTTCTT TTACTCCCCC | 3250 |
| CACCGCCCCC GCTCTTTCTC TGTCTCTGTT TTTTTCTTTT CCAGAAAATC | 3300 |
| AGTTCGAGGA AAGGGAAAGG GGCAAAAACG AAAGCGCAAG AAATCTAGAT | 3350 |
| ATAAGTCCTG GAGCGTGTAA GTTGGTGCCC GCTGCTGTCT AATGCCCTGG | 3400 |
| AGCCTCCCTG GCCCCCAGTA CAACCTCCGC CTGCCATTCC CTGTAACCCT | 3450 |
| GCCTCCCTCC CCTGGTCCCT CCCTGGCTCT CATCCTCCTG GCCCGTGTCT | 3500 |
| CTCTCTCACT CTCTCACTCC ACTAATTGGC ACCAACGGGT AGATTTGGTG | 3550 |
| GTGGCATTGC TGGTCCAGGG TTGGGGTGAA TGGGGGTGCC GACTTGGCCT | 3600 |
| GGAGGATTAA GGGAGGGGAC CCTGGCTTGG CTGGGCACCG ATTTTCTCTC | 3650 |
| ACCCACTGGG CACTGGTGGC AGGCCCATGT TGGCACAGGT GCCTGCTCAC | 3700 |
| CCAACTGGTT TCCATTGCTC TAGGCTTCTG CACTCGTCTG GAAGCTGAGG | 3750 |
| GTGGTGGGGA GGGCAGACAT GGCCCAAGAA GGGCTGTGAA TGACTGGAGG | 3800 |
| CAGCTTGCTG AATGACTCCT TGGCTGAAGG AGGAGCTTGG GTGGGATCAG | 3850 |
| ACACCATGTG GCGGCCTCCC TTCATCTGGT GGAAGTGCCC TGGCTCCTCA | 3900 |
| CGGAGGTGGG GCCTCTGGAG GGGAGCCCCC TATTCTGGCC CAACCCATGG | 3950 |
| CACCCACAGA GGCCTCCTTG CAGGGCAGCC TCTTCCTCCG GGTCGGAGGC | 4000 |
| TGTGGTGGGC CCTGCCCTGG GCCCTCTGGC CACCAGCGGC CTGGCCTGGG | 4050 |
| GACACTGCCT CCGGGCTTAG CCTCCCATCA CACCCTACTT TAGCCCACCT | 4100 |
| TGGTGGAAGG GCCTGGACAT GAGCCTTGCA CGGGGAGAAG GTGGCCCCTG | 4150 |
| ATTGCCATCC CCAGCAGGTG AAGAGTCAAG GCGTGCTCCG ATGGGGGCAA | 4200 |
| CAGCAGTTGG GTCCCTGTGG CCTGAGACTC ACCCTTGTCT CCCAGAGACA | 4250 |
| CAGCATTGCC CCTTATGGCA GCCTCTCCCT GCACTCTCTG CCCGTCTGTG | 4300 |
| CCCGCCTCTT CCTGCGGCAG GTGTCCTAGC CAGTGCTGCC TCTTTCCGCC | 4350 |
| GCTCTCTCTG TCTTTTGCTG TAGCGCTCGG ATCCTTCCAG GGCCTGGGGG | 4400 |
| CTGACCGGCT GGGTGGGGGT GCAGCTGCGG ACATGTTAGG GGGTGTTGCA | 4450 |
| TGGTGATTTT TTTTCTCTCT CTCTGCTGAT GCTCTAGCTT AGATGTCTTT | 4500 |
| CCTTTTGCCT TTTTGCAGTC CCTGTGGGCC TTGCTCAGAG CGGAGAAAGC | 4550 |
| ATTTGTTTGT ACAAGATCCG CAGACGTGTA AATGTTCCTG CAAAAACACA | 4600 |
| GACTCGAGAT GCAAGGCGAG GCAGCTTGAG TTAAACGAAC GTACTTGCAG | 4650 |
| GTTGGTTCCC AGAGGGCAAG CAAGTCAGAG AGGGGCATCA CACAGAGATG | 4700 |
| GGGAGAGAGA GAGAGAAAGA GAGTGAGCGA GCGAGCGAGC GGGAGAGCGC | 4750 |
| CTGAGAGGGG CCAGCTGCTT GCTCAGTTTC TAGCTGCCTG CCTGAGATCT | 4800 |
| GCGAAGGGCG AATTCCAGCA CACTGGCGGC CGTTACTAGT GGATCTGCCC | 4850 |
| ACTCTCTTCC CCACACCAGC CCCTAGAGAC TGAACTGAAA ACCCTCCTCA | 4900 |
| GCAGGGAGCC TCTTCTGATT AACTTCATCC AGCTCTGGTC ACCCATCAGC | 4950 |
| TCTTAAAATG TCAAGTGGGG ACTGTTCTTT GGTATCCGTT CATTTGTTGC | 5000 |
| TTTGTAAAGT GTTCCCATGT CCTTGTCTTG TCTCAAGTAG ATTGCAAGCT | 5050 |
| CAGGAGGGTA GACTGGGAGC CCCTGAGTGG AGCTGCTGCT CAGGCCGGGG | 5100 |
| CTCCCTGAGG GCAGGGCTGG GGCTGTTCTC ATACTGGGGC TTTCTGCCCC | 5150 |
| AGGACCACAC CTTCCTGTCC CCTCTGCTCT TATGGTGCCG GAGGCTGCAG | 5200 |
| TGACCCAGGG GCCCCCAGGA ATGGGGAGGC CGCCTGCCTC ATCGCCAGGC | 5250 |
| CTCCTCACTT GGCCCTAACC CCAGCCTTTG TTTTCCATTT CCCTCAGATG | 5300 |
| TGACAAGCCG AGGCGGTGAA AGCTTCTAGA TAAGATATCC GATCCACCGG | 5350 |
| ATCTAGATAA CTGATCATAA TCAGCCATAC CACATTTGTA GAGGTTTTAC | 5400 |
| TTGCTTTAAA AAACCTCCCA CACCTCCCCC TGAACCTGAA ACATAAAATG | 5450 |
| AATGCAATTG TTGTTGTTAA CTTGTTTATT GCAGCTTATA ATGGTTACAA | 5500 |
| ATAAAGCAAT AGCATCACAA ATTTCACAAA TAAAGCATTT TTTTCACTGC | 5550 |
| ATTCTAGTTG TGGTTTGTCC AAACTCATCA ATGTATCTTA ACGCGGATCT | 5600 |
| GGGCGTGGTT AAGGGTGGGA AAGAATATAT AAGGTGGGGG TCTTATGTAG | 5650 |
| TTTTGTATCT GTTTTGCAGC AGCCGCCGCC GCCATGAGCA CCAACTCGTT | 5700 |
| TGATGGAAGC ATTGTGAGCT CATATTTGAC AACGCGCATG CCCCCATGGG | 5750 |
| CCGGGGTGCG TCAGAATGTG ATGGGCTCCA GCATTGATGG TCGCCCCGTC | 5800 |
| CTGCCCGCAA ACTCTACTAC CTTGACCTAC GAGACCGTGT CTGGAACGCC | 5850 |
| GTTGGAGACT GCAGCCTCCG CCGCCGCTTC AGCCGCTGCA GCCACCGCCC | 5900 |
| GCGGGATTGT GACTGACTTT GCTTTCCTGA GCCCGCTTGC AAGCAGTGCA | 5950 |
| GCTTCCCGTT CATCCGCCCG CGATGACAAG TTGACGGCTC TTTTGGCACA | 6000 |
| ATTGGATTCT TTGACCCGGG AACTTAATGT CGTTTCTCAG CAGCTGTTGG | 6050 |
| ATCTGCGCCA GCAGGTTTCT GCCCTGAAGG CTTCCTCCCC TCCCAATGCG | 6100 |
| GTTTAAAACA TAAATAAAAA ACCAGACTCT GTTTGGATTT GGATCAAGCA | 6150 |
| AGTGTCTTGC TGTCTTTATT TAGGGGTTTT GCGCGCGCGG TAGGCCCGGG | 6200 |
| ACCAGCGGTC TCGGTCGTTG AGGGTCCTGT GTATTTTTTC CAGGACGTGG | 6250 |
| TAAAGGTGAC TCTGGATGTT CAGATACATG GGCATAAGCC CGTCTCTGGG | 6300 |
| GTGGAGGTAG CACCACTGCA GAGCTTCATG CTGCGGGGTG GTGTTGTAGA | 6350 |
| TGATCCAGTC GTAGCAGGAG CGCTGGGCGT GGTGCCTAAA AATGTCTTTC | 6400 |
| AGTAGCAAGC TGATTGCCAG GGGCAGGCCC TTGGTGTAAG TGTTTACAAA | 6450 |
| GCGGTTAAGC TGGGATGGGT GCATACGTGG GGATATGAGA TGCATCTTGG | 6500 |
| ACTGTATTTT TAGGTTGGCT ATGTTCCCAG CCATATCCCT CCGGGGATTC | 6550 |
| ATGTTGTGCA GAACCACCAG CACAGTGTAT CCGGTGCACT TGGGAAATTT | 6600 |
| GTCATGTAGC TTAGAAGGAA ATGCGTGGAA GAACTTGGAG ACGCCCTTGT | 6650 |
| GACCTCCAAG ATTTTCCATG CATTCGTCCA TAATGATGGC AATGGGCCCA | 6700 |
| CGGGCGGCGG CCTGGGCGAA GATATTTCTG GGATCACTAA CGTCATAGTT | 6750 |
| GTGTTCCAGG ATGAGATCGT CATAGGCCAT TTTTACAAAG CGCGGGCGGA | 6800 |
| GGGTGCCAGA CTGCGGTATA ATGGTTCCAT CCGGCCCAGG GGCGTAGTTA | 6850 |
| CCCTCACAGA TTTGCATTTC CCACGCTTTG AGTTCAGATG GGGGGATCAT | 6900 |
| GTCTACCTGC GGGGCGATGA AGAAAACGGT TTCCGGGGTA GGGGAGATCA | 6950 |
| GCTGGGAAGA AAGCAGGTTC CTGAGCAGCT GCGACTTACC GCAGCCGGTG | 7000 |
| GGCCCGTAAA TCACACCTAT TACCGGCTGC AACTGGTAGT TAAGAGAGCT | 7050 |
| GCAGCTGCCG TCATCCCTGA GCAGGGGGGC CACTTCGTTA AGCATGTCCC | 7100 |
| TGACTCGCAT GTTTTCCCTG ACCAAATCCG CCAGAAGGCG CTCGCCGCCC | 7150 |
| AGCGATAGCA GTTCTTGCAA GGAAGCAAAG TTTTTCAACG GTTTGAGACC | 7200 |
| GTCCGCCGTA GGCATGCTTT TGAGCGTTTG ACCAAGCAGT TCCAGGCGGT | 7250 |
| CCCACAGCTC GGTCACCTGC TCTACGGCAT CTCGATCCAG CATATCTCCT | 7300 |
| CGTTTCGCGG GTTGGGGCGG CTTTCGCTGT ACGGCAGTAG TCGGTGCTCG | 7350 |
| TCCAGACGGG CCAGGGTCAT GTCTTTCCAC GGGCGCAGGG TCCTCGTCAG | 7400 |
| CGTAGTCTGG GTCACGGTGA AGGGGTGCGC TCCGGGCTGC GCGCTGGCCA | 7450 |
| GGGTGCGCTT GAGGCTGGTC CTGCTGGTGC TGAAGCGCTG CCGGTCTTCG | 7500 |
| CCCTGCGCGT CGGCCAGGTA GCATTTGACC ATGGTGTCAT AGTCCAGCCC | 7550 |
| CTCCGCGGCG TGGCCCTTGG CGCGCAGCTT GCCCTTGGAG GAGGCGCCGC | 7600 |
| ACGAGGGGCA GTGCAGACTT TTGAGGGCGT AGAGCTTGGG CGCGAGAAAT | 7650 |
| ACCGATTCCG GGGAGTAGGC ATCCGCGCCG CAGGCCCCGC AGACGGTCTC | 7700 |
| GCATTCCACG AGCCAGGTGA GCTCTGGCCG TTCGGGGTCA AAAACCAGGT | 7750 |
| TTCCCCCATG CTTTTTGATG CGTTTCTTAC CTCTGGTTTC CATGAGCCGG | 7800 |
| TGTCCACGCT CGGTGACGAA AAGGCTGTCC GTGTCCCCGT ATACAGACTT | 7850 |
| GAGAGGCCTG TCCTCGAGCG GTGTTCCGCG GTCCTCCTCG TATAGAAACT | 7900 |
| CGGACCACTC TGAGACAAAG GCTCGCGTCC AGGCCAGCAC GAAGGAGGCT | 7950 |
| AAGTGGGAGG GGTAGCGGTC GTTGTCCACT AGGGGGTCCA CTCGCTCCAG | 8000 |
| GGTGTGAAGA CACATGTCGC CCTCTTCGGC ATCAAGGAAG GTGATTGGTT | 8050 |
| TGTAGGTGTA GGCCACGTGA CCGGGTGTTC CTGAAGGGGG GCTATAAAAG | 8100 |
| GGGGTGGGGG CGCGTTCGTC CTCACTCTCT TCCGCATCGC TGTCTGCGAG | 8150 |
| GGCCAGCTGT TGGGGTGAGT ACTCCCTCTG AAAAGCGGGC ATGACTTCTG | 8200 |
| CGCTAAGATT GTCAGTTTCC AAAAACGAGG AGGATTTGAT ATTCACCTGG | 8250 |
| CCCGCGGTGA TGCCTTTGAG GGTGGCCGCA TCCATCTGGT CAGAAAAGAC | 8300 |
| AATCTTTTTG TTGTCAAGCT TGGTGGCAAA CGACCCGTAG AGGGCGTTGG | 8350 |
| ACAGCAACTT GGCGATGGAG CGCAGGGITT GGTTTTTGTC GCGATCGGCG | 8400 |
| CGCTCCTTGG CCGCGATGTT TAGCTGCACG TATTCGCGCG CAACGCACCG | 8450 |
| CCATTCGGGA AAGACGGTGG TGCGCTCGTC GGGCACCAGG TGCACGCGCC | 8500 |
| AACCGCGGTT GTGCAGGGTG ACAAGGTCAA CGCTGGTGGC TACCTCTCCG | 8550 |
| CGTAGGCGCT CGTTGGTCCA GCAGAGGCGG CCGCCCTTGC GCGAGCAGAA | 8600 |
| TGGCGGTAGG GGGTCTAGCT GCGTCTCGTC CGGGGGGTCT GCGTCCACGG | 8650 |
| TAAAGACCCC GGGCAGCAGG CGCGCGTCGA AGTAGTCTAT CTTGCATCCT | 8700 |
| TGCAAGTCTA GCGCCTGCTG CCATGCGCGG GCGGCAAGCG CGCGCTCGTA | 8750 |
| TGGGTTGAGT GGGGGACCCC ATGGCATGGG GTGGGTGAGC GCGGAGGCGT | 8800 |
| ACATGCCGCA AATGTCGTAA ACGTAGAGGG GCTCTCTGAG TATTCCAAGA | 8850 |
| TATGTAGGGT AGCATCTTCC ACCGCGGATG CTGGCGCGCA CGTAATCGTA | 8900 |
| TAGTTCGTGC GAGGGAGCGA GGAGGTCGGG ACCGAGGITG CTACGGGCGG | 8950 |
| GCTGCTCTGC TCGGAAGACT ATCTGCCTGA AGATGGCATG TGAGTTGGAT | 9000 |
| GATATGGTTG GACGCTGGAA GACGTTGAAG CTGGCGTCTG TGAGACCTAC | 9050 |
| CGCGTCACGC ACGAAGGAGG CGTAGGAGTC GCGCAGCTTG TTGACCAGCT | 9100 |
| CGGCGGTGAC CTGCACGTCT AGGGCGCAGT AGTCCAGGGT TTCCTTGATG | 9150 |
| ATGTCATACT TATCCTGTCC CTTTTTTTTC CACAGCTCGC GGTTGAGGAC | 9200 |
| AAACTCTTCG CGGTCTTTCC AGTACTCTTG GATCGGAAAC CCGTCGGCCT | 9250 |
| CCGAACGGTA AGAGCCTAGC ATGTAGAACT GGTTGACGGC CTGGTAGGCG | 9300 |
| CAGCATCCCT TTTCTACGGG TAGCGCGTAT GCCTGCGCGG CCTTCCGGAG | 9350 |
| CGAGGTGTGG GTGAGCGCAA AGGTGTCCCT GACCATGACT TTGAGGTACT | 9400 |
| GGTATTTGAA GTCAGTGTCG TCGCATCCGC CCTGCTCCCA GAGCAAAAAG | 9450 |
| TCCGTGCGCT TTTTGGAACG CGGATTTGGC AGGGCGAAGG TGACATCGTT | 9500 |
| GAAGAGTATC TTTCCCGCGC GAGGCATAAA GTTGCGTGTG ATGCGGAAGG | 9550 |
| GTCCCGGCAC CTCGGAACGG TTGTTAATTA CCTGGGCGGC GAGCACGATC | 9600 |
| TCGTCAAAGC CGTTGATGTT GTGGCCCACA ATGTAAAGTT CCAAGAAGCG | 9650 |
| CGGGATGCCC TTGATGGAAG GCAATTTTTT AAGTTCCTCG TAGGTGAGCT | 9700 |
| CTTCAGGGGA GCTGAGCCCG TGCTCTGAAA GGGCCCAGTC TGCAAGATGA | 9750 |
| GGGTTGGAAG CGACGAATGA GCTCCACAGG TCACGGGCCA TTAGCATTTG | 9800 |
| CAGGTGGTCG CGAAAGGTCC TAAACTGGCG ACCTATGGCC ATTTTTTCTG | 9850 |
| GGGTGATGCA GTAGAAGGTA AGCGGGTCTT GTTCCCAGCG GTCCCATCCA | 9900 |
| AGGTTCGCGG CTAGGTCTCG CGCGGCAGTC ACTAGAGGCT CATCTCCGCC | 9950 |
| GAACTTCATG ACCAGCATGA AGGGCACGAG CTGCTTCCCA AAGGCCCCCA | 10000 |
| TCCAAGTATA GGTCTCTACA TCGTAGGTGA CAAAGAGACG CTCGGTGCGA | 10050 |
| GGATGCGAGC CGATCGGGAA GAACTGGATC TCCCGCCACC AATTGGAGGA | 10100 |
| GTGGCTATTG ATGTGGTGAA AGTAGAAGTC CCTGCGACGG GCCGAACACT | 10150 |
| CGTGCTGGCT TTTGTAAAAA CGTGCGCAGT ACTGGCAGCG GTGCACGGGC | 10200 |
| TGTACATCCT GCACGAGGTT GACCTGACGA CCGCGCACAA GGAAGCAGAG | 10250 |
| TGGGAATTTG AGCCCCTCGC CTGGCGGGTT TGGCTGGTGG TCTTCTACTT | 10300 |
| CGGCTGCTTG TCCTTGACCG TCTGGCTGCT CGAGGGGAGT TACGGTGGAT | 10350 |
| CGGACCACCA CGCCGCGCGA GCCCAAAGTC CAGATGTCCG CGCGCGGCGG | 10400 |
| TCGGAGCTTG ATGACAACAT CGCGCAGATG GGAGCTGTCC ATGGTCTGGA | 10450 |
| GCTCCCGCGG CGTCAGGTCA GGCGGGAGCT CCTGCAGGTT TACCTCGCAT | 10500 |
| AGACGGGTCA GGGCGCGGGC TAGATCCAGG TGATACCTAA TTTCCAGGGG | 10550 |
| CTGGTTGGTG GCGGCGTCGA TGGCTTGCAA GAGGCCGCAT CCCCGCGGCG | 10600 |
| CGACTACGGT ACCGCGCGGC GGGCGGTGGG CCGCGGGGGT GTCCTTGGAT | 10650 |
| GATGCATCTA AAAGCGGTGA CGCGGGCGAG CCCCCGGAGG TAGGGGGGGC | 10700 |
| TCCGGACCCG CCGGGAGAGG GGGCAGGGGC ACGTCGGCGC CGCGCGCGGG | 10750 |
| CAGGAGCTGG TGCTGCGCGC GTAGGTTGCT GGCGAACGCG ACGACGCGGC | 10800 |
| GGTTGATCTC CTGAATCIGG CGCCTCTGCG TGAAGACGAC GGGCCCGGTG | 10850 |
| AGCTTGAACC TGAAAGAGAG TTCGACAGAA TCAATTTCGG TGTCGTTGAC | 10900 |
| GGCGGCCTGG CGCAAAATCT CCTGCACGTC TCCTGAGTTG TCTTGATAGG | 10950 |
| CGATCTCGGC CATGAACTGC TCGATCTCTT CCTCCTGGAG ATCTCCGCGT | 11000 |
| CCGGCTCGCT CCACGGTGGC GGCGAGGICG TTGGAAATGC GGGCCATGAG | 11050 |
| CTGCGAGAAG GCGTTGAGGC CTCCCTCGTT CCAGACGCGG CTGTAGACCA | 11100 |
| CGCCCCCTTC GGCATCGCGG GCGCGCATGA CCACCTGCGC GAGATTGAGC | 11150 |
| TCCACGTGCC GGGCGAAGAC GGCGTAGTTT CGCAGGCGCT GAAAGAGGTA | 11200 |
| GTTGAGGGTG GTGGCGGTGT GTTCTGCCAC GAAGAAGTAC ATAACCCAGC | 11250 |
| GTCGCAACGT GGATTCGTTG ATATCCCCCA AGGCCTCAAG GCGCTCCATG | 11300 |
| GCCTCGTAGA AGTCCACGGC GAAGTTGAAA AACTGGGAGT TGCGCGCCGA | 11350 |
| CACGGTTAAC TCCTCCTCCA GAAGACGGAT GAGCTCGGCG ACAGTGTCGC | 11400 |
| GCACCTCGCG CTCAAAGGCT ACAGGGGCCT CTTCTTCTTC TTCAATCTCC | 11450 |
| TCTTCCATAA GGGCCTCCCC TTCTTCTTCT TCTGGCGGCG GTGGGGGAGG | 11500 |
| GGGGACACGG CGGCGACGAC GGCGCACCGG GAGGCGGTCG ACAAAGCGCT | 11550 |
| CGATCATCTC CCCGCGGCGA CGGCGCATGG TCTCGGTGAC GGCGCGGCCG | 11600 |
| TTCTCGCGGG GGCGCAGTTG GAAGACGCCG CCCGTCATGT CCCGGTTATG | 11650 |
| GGTTGGCGGG GGGCTGCCAT GCGGCAGGGA TACGGCGCTA ACGATGCATC | 11700 |
| TCAACAATTG TTGTGTAGGT ACTCCGCCGC CGAGGGACCT GAGCGAGTCC | 11750 |
| GCATCGACCG GATCGGAAAA CCTCTCGAGA AAGGCGTCTA ACCAGTCACA | 11800 |
| GTCGCAAGGT AGGCTGAGCA CCGTGGCGGG CGGCAGCGGG CGGCGGTCGG | 11850 |
| GGTTGTTTCT GGCGGAGGTG CTGCTGATGA TGTAATTAAA GTAGGCGGTC | 11900 |
| TTGAGACGGC GGATGGTCGA CAGAAGCACC ATGTCCTTGG GTCCGGCCTG | 11950 |
| CTGAATGCGC AGGCGGTCGG CCATGCCCCA GGCTTCGTTT TGACATCGGC | 12000 |
| GCAGGTCTTT GTAGTAGTCT TGCATGAGCC TTTCTACCGG CACTTCTTCT | 12050 |
| TCTCCTTCCT CTTGTCCTGC ATCTCTTGCA TCTATCGCTG CGGCGGCGGC | 12100 |
| GGAGTTTGGC CGTAGGTGGC GCCCTCTTCC TCCCATGCGT GTGACCCCGA | 12150 |
| AGCCCCTCAT CGGCTGAAGC AGGGCTAGGT CGGCGACAAC GCGCTCGGCT | 12200 |
| AATATGGCCT GCTGCACCTG CGTGAGGGTA GACTGGAAGT CATCCATGTC | 12250 |
| CACAAAGCGG TGGTATGCGC CCGTGTTGAT GGTGTAAGTG CAGTTGGCCA | 12300 |
| TAACGGACCA GTTAACGGTC TGGTGACCCG GCTGCGAGAG CTCGGTGTAC | 12350 |
| CTGAGACGCG AGTAAGCCCT CGAGTCAAAT ACGTAGTCGT TGCAAGTCCG | 12400 |
| CACCAGGTAC TGGTATCCCA CCAAAAAGTG CGGCGGCGGC TGGCGGTAGA | 12450 |
| GGGGCCAGCG TAGGGTGGCC GGGGCTCCGG GGGCGAGATC TTCCAACATA | 12500 |
| AGGCGATGAT ATCCGTAGAT GTACCTGGAC ATCCAGGTGA TGCCGGCGGC | 12550 |
| GGTGGTGGAG GCGCGCGGAA AGTCGCGGAC GCGGTTCCAG ATGTTGCGCA | 12600 |
| GCGGCAAAAA GTGCTCCATG GTCGGGACGC TCTGGCCGGT CAGGCGCGCG | 12650 |
| CAATCGTTGA CGCTCTAGCG TGCAAAAGGA GAGCCTGTAA GCGGGCACTC | 12700 |
| TTCCGTGGTC TGGTGGATAA ATTCGCAAGG GTATCATGGC GGACGACCGG | 12750 |
| GGTTCGAGCC CCGTATCCGG CCGTCCGCCG TGATCCATGC GGTTACCGCC | 12800 |
| CGCGTGTCGA ACCCAGGTGT GCGACGTCAG ACAACGGGGG AGTGCTCCTT | 12850 |
| TTGGCTTCCT TCCAGGCGCG GCGGCTGCTG CGCTAGCTTT TTTGGCCACT | 12900 |
| GGCCGCGCGC AGCGTAAGCG GTTAGGCTGG AAAGCGAAAG CATTAAGTGG | 12950 |
| CTCGCTCCCT GTAGCCGGAG GGTTATTTTC CAAGGGTTGA GTCGCGGGAC | 13000 |
| CCCCGGTTCG AGTCTCGGAC CGGCCGGACT GCGGCGAACG GGGGTTTGCC | 13050 |
| TCCCCGTCAT GCAAGACCCC GCTTGCAAAT TCCTCCGGAA ACAGGGACGA | 13100 |
| GCCCCTTTTT TGCTTTTCCC AGATGCATCC GGTGCTGCGG CAGATGCGCC | 13150 |
| CCCCTCCTCA GCAGCGGCAA GAGCAAGAGC AGCGGCAGAC ATGCAGGGCA | 13200 |
| CCCTCCCCTC CTCCTACCGC GTCAGGAGGG GCGACATCCG CGGTTGACGC | 13250 |
| GGCAGCAGAT GGTGATTACG AACCCCCGCG GCGCCGGGCC CGGCACTACC | 13300 |
| TGGACTTGGA GGAGGGCGAG GGCCTGGCGC GGCTAGGAGC GCCCTCTCCT | 13350 |
| GAGCGGCACC CAAGGGTGCA GCTGAAGCGT GATACGCGTG AGGCGTACGT | 13400 |
| GCCGCGGCAG AACCTGTTTC GCGACCGCGA GGGAGAGGAG CCCGAGGAGA | 13450 |
| TGCGGGATCG AAAGTTCCAC GCAGGGCGCG AGCTGCGGCA TGGCCTGAAT | 13500 |
| CGCGAGCGGT TGCTGCGCGA GGAGGACTTT GAGCCCGACG CGCGAACCGG | 13550 |
| GATTAGTCCC GCGCGCGCAC ACGTGGCGGC CGCCGACCTG GTAACCGCAT | 13600 |
| ACGAGCAGAC GGTGAACCAG GAGATTAACT TTCAAAAAAG CTTTAACAAC | 13650 |
| CACGTGCGTA CGCTTGTGGC GCGCGAGGAG GTGGCTATAG GACTGATGCA | 13700 |
| TCTGTGGGAC TTTGTAAGCG CGCTGGAGCA AAACCCAAAT AGCAAGCCGC | 13750 |
| TCATGGCGCA GCTGTTCCTT ATAGTGCAGC ACAGCAGGGA CAACGAGGCA | 13800 |
| TTCAGGGATG CGCTGCTAAA CATAGTAGAG CCCGAGGGCC GCTGGCTGCT | 13850 |
| CGATTTGATA AACATCCTGC AGAGCATAGT GGTGCAGGAG CGCAGCTTGA | 13900 |
| GCCTGGCTGA CAAGGTGGCC GCCATCAACT ATTCCATGCT TAGCCTGGGC | 13950 |
| AAGTTTTACG CCCGCAAGAT ATACCATACC CCTTACGTTC CCATAGACAA | 14000 |
| GGAGGTAAAG ATCGAGGGGT TCTACATGCG CATGGCGCTG AAGGTGCTTA | 14050 |
| CCTTGAGCGA CGACCTGGGC GTTTATCGCA ACGAGCGCAT CCACAAGGCC | 14100 |
| GTGAGCGTGA GCCGGCGGCG CGAGCTCAGC GACCGCGAGC TGATGCACAG | 14150 |
| CCTGCAAAGG GCCCTGGCTG GCACGGGCAG CGGCGATAGA GAGGCCGAGT | 14200 |
| CCTACTTTGA CGCGGGCGCT GACCTGCGCT GGGCCCCAAG CCGACGCGCC | 14250 |
| CTGGAGGCAG CTGGGGCCGG ACCTGGGCTG GCGGTGGCAC CCGCGCGCGC | 14300 |
| TGGCAACGTC GGCGGCGTGG AGGAATATGA CGAGGACGAT GAGTACGAGC | 14350 |
| CAGAGGACGG CGAGTACTAA GCGGTGATGT TTCTGATCAG ATGATGCAAG | 14400 |
| ACGCAACGGA CCCGGCGGTG CGGGCGGCGC TGCAGAGCCA GCCGTCCGGC | 14450 |
| CTTAACTCCA CGGACGACTG GCGCCAGGTC ATGGACCGCA TCATGTCGCT | 14500 |
| GACTGCGCGC AATCCTGACG CGTTCCGGCA GCAGCCGCAG GCCAACCGGC | 14550 |
| TCTCCGCAAT TCTGGAAGCG GTGGTCCCGG CGCGCGCAAA CCCCACGCAC | 14600 |
| GAGAAGGTGC TGGCGATCGT AAACGCGCTG GCCGAAAACA GGGCCATCCG | 14650 |
| GCCCGACGAG GCCGGCCTGG TCTACGACGC GCTGCTTCAG CGCGTGGCTC | 14700 |
| GTTACAACAG CGGCAACGTG CAGACCAACC TGGACCGGCT GGTGGGGGAT | 14750 |
| GTGCGCGAGG CCGTGGCGCA GCGTGAGCGC GCGCAGCAGC AGGGCAACCT | 14800 |
| GGGCTCCATG GTTGCACTAA ACGCCTTCCT GAGTACACAG CCCGCCAACG | 14850 |
| TGCCGCGGGG ACAGGAGGAC TACACCAACT TTGTGAGCGC ACTGCGGCTA | 14900 |
| ATGGTGACTG AGACACCGCA AAGTGAGGTG TACCAGTCTG GGCCAGACTA | 14950 |
| TTTTTTCCAG ACCAGTAGAC AAGGCCTGCA GACCGTAAAC CTGAGCCAGG | 15000 |
| CTTTCAAAAA CTTGCAGGGG CTGTGGGGGG TGCGGGCTCC CACAGGCGAC | 15050 |
| CGCGCGACCG TGTCTAGCTT GCTGACGCCC AACTCGCGCC TGTTGCTGCT | 15100 |
| GCTAATAGCG CCCTTCACGG ACAGTGGCAG CGTGTCCCGG GACACATACC | 15150 |
| TAGGTCACTT GCTGACACTG TACCGCGAGG CCATAGGTCA GGCGCATGTG | 15200 |
| GACGAGCATA CTTTCCAGGA GATTACAAGT GTCAGCCGCG CGCTGGGGCA | 15250 |
| GGAGGACACG GGCAGCCTGG AGGCAACCCT AAACTACCTG CTGACCAACC | 15300 |
| GGCGGCAGAA GATCCCCTCG TTGCACAGTT TAAACAGCGA GGAGGAGCGC | 15350 |
| ATTTTGCGCT ACGTGCAGCA GAGCGTGAGC CTTAACCTGA TGCGCGACGG | 15400 |
| GGTAACGCCC AGCGTGGCGC TGGACATGAC CGCGCGCAAC ATGGAACCGG | 15450 |
| GCATGTATGC CTCAAACCGG CCGTTTATCA ACCGCCTAAT GGACTACTTG | 15500 |
| CATCGCGCGG CCGCCGTGAA CCCCGAGTAT TTCACCAATG CCATCTTGAA | 15550 |
| CCCGCACTGG CTACCGCCCC CTGGTTTCTA CACCGGGGGA TTCGAGGTGC | 15600 |
| CCGAGGGTAA CGATGGATTC CTCTGGGACG ACATAGACGA CAGCGTGTTT | 15650 |
| TCCCCGCAAC CGCAGACCCT GCTAGAGTTG CAACAGCGCG AGCAGGCAGA | 15700 |
| GGCGGCGCTG CGAAAGGAAA GCTTCCGCAG GCCAAGCAGC TTGTCCGATC | 15750 |
| TAGGCGCTGC GGCCCCGCGG TCAGATGCTA GTAGCCCATT TCCAAGCTTG | 15800 |
| ATAGGGTCTC TTACCAGCAC TCGCACCACC CGCCCGCGCC TGCTGGGCGA | 15850 |
| GGAGGAGTAC CTAAACAACT CGCTGCTGCA GCCGCAGCGC GAAAAAAACC | 15900 |
| TGCCTCCGGC ATTTCCCAAC AACGGGATAG AGAGCCTAGT GGACAAGATG | 15950 |
| AGTAGATGGA AGACGTACGC GCAGGAGCAC AGGGACGTGC CAGGCCCGCG | 16000 |
| CCCGCCCACC CGTCGTCAAA GGCACGACCG TCAGCGGGGT CTGGTGTGGG | 16050 |
| AGGACGATGA CTCGGCAGAC GACAGCAGCG TCCTGGATTT GGGAGGGAGT | 16100 |
| GGCAACCCGT TTGCGCACCT TCGCCCCAGG CTGGGGAGAA TGTTTTAAAA | 16150 |
| AAAAAAAAGC ATGATGCAAA ATAAAAAACT CACCAAGGCC ATGGCACCGA | 16200 |
| GCGTTGGTTT TCTTGTATTC CCCTTAGTAT GCGGCGCGCG GCGATGTATG | 16250 |
| AGGAAGGTCC TCCTCCCTCC TACGAGAGTG TGGTGAGCGC GGCGCCAGTG | 16300 |
| GCGGCGGCGC TGGGTTCTCC CTTCGATGCT CCCCTGGACC CGCCGTTTGT | 16350 |
| GCCTCCGCGG TACCTGCGGC CTACCGGGGG GAGAAACAGC ATCCGTTACT | 16400 |
| CTGAGTTGGC ACCCCTATTC GACACCACCC GTGTGTACCT GGTGGACAAC | 16450 |
| AAGTCAACGG ATGTGGCATC CCTGAACTAC CAGAACGACC ACAGCAACTT | 16500 |
| TCTGACCACG GTCATTCAAA ACAATGACTA CAGCCCGGGG GAGGCAAGCA | 16550 |
| CACAGACCAT CAATCTTGAC GACCGGTCGC ACTGGGGCGG CGACCTGAAA | 16600 |
| ACCATCCTGC ATACCAACAT GCCAAATGTG AACGAGTTCA TGTTTACCAA | 16650 |
| TAAGTTTAAG GCGCGGGTGA TGGTGTCGCG CTTGCCTACT AAGGACAATC | 16700 |
| AGGTGGAGCT GAAATACGAG TGGGTGGAGT TCACGCTGCC CGAGGGCAAC | 16750 |
| TACTCCGAGA CCATGACCAT AGACCTTATG AACAACGCGA TCGTGGAGCA | 16800 |
| CTACTTGAAA GTGGGCAGAC AGAACGGGGT TCTGGAAAGC GACATCGGGG | 16850 |
| TAAAGTTTGA CACCCGCAAC TTCAGACTGG GGTTTGACCC CGTCACTGGT | 16900 |
| CTTGTCATGC CTGGGGTATA TACAAACGAA GCCTTCCATC CAGACATCAT | 16950 |
| TTTGCTGCCA GGATGCGGGG TGGACTTCAC CCACAGCCGC CTGAGCAACT | 17000 |
| TGTTGGGCAT CCGCAAGCGG CAACCCTTCC AGGAGGGCTT TAGGATCACC | 17050 |
| TACGATGATC TGGAGGGTGG TAACATTCCC GCACTGTTGG ATGTGGACGC | 17100 |
| CTACCAGGCG AGCTTGAAAG ATGACACCGA ACAGGGCGGG GGTGGCGCAG | 17150 |
| GCGGCAGCAA CAGCAGTGGC AGCGGCGCGG AAGAGAACTC CAACGCGGCA | 17200 |
| GCCGCGGCAA TGCAGCCGGT GGAGGACATG AACGATCATG CCATTCGCGG | 17250 |
| CGACACCTTT GCCACACGGG CTGAGGAGAA GCGCGCTGAG GCCGAAGCAG | 17300 |
| CGGCCGAAGC TGCCGCCCCC GCTGCGCAAC CCGAGGTCGA GAAGCCTCAG | 17350 |
| AAGAAACCGG TGATCAAACC CCTGACAGAG GACAGCAAGA AACGCAGTTA | 17400 |
| CAACCTAATA AGCAATGACA GCACCTTCAC CCAGTACCGC AGCTGGTACC | 17450 |
| TTGCATACAA CTACGGCGAC CCTCAGACCG GAATCCGCTC ATGGACCCTG | 17500 |
| CTTTGCACTC CTGACGTAAC CTGCGGCTCG GAGCAGGTCT ACTGGTCGTT | 17550 |
| GCCAGACATG ATGCAAGACC CCGTGACCTT CCGCTCCACG CGCCAGATCA | 17600 |
| GCAACTTTCC GGTGGTGGGC GCCGAGCTGT TGCCCGTGCA CTCCAAGAGC | 17650 |
| TTCTACAACG ACCAGGCCGT CTACTCCCAA CTCATCCGCC AGTTTACCTC | 17700 |
| TCTGACCCAC GTGTTCAATC GCTTTCCCGA GAACCAGATT TTGGCGCGCC | 17750 |
| CGCCAGCCCC CACCATCACC ACCGTCAGTG AAAACGTTCC TGCTCTCACA | 17800 |
| GATCACGGGA CGCTACCGCT GCGCAACAGC ATCGGAGGAG TCCAGCGAGT | 17850 |
| GACCATTACT GACGCCAGAC GCCGCACCTG CCCCTACGTT TACAAGGCCC | 17900 |
| TGGGCATAGT CTCGCCGCGC GTCCTATCGA GCCGCACTTT TTGAGCAAGC | 17950 |
| ATGTCCATCC TTATATCGCC CAGCAATAAC ACAGGCTGGG GCCTGCGCTT | 18000 |
| CCCAAGCAAG ATGTTTGGCG GGGCCAAGAA GCGCTCCGAC CAACACCCAG | 18050 |
| TGCGCGTGCG CGGGCACTAC CGCGCGCCCT GGGGCGCGCA CAAACGCGGC | 18100 |
| CGCACTGGGC GCACCACCGT CGATGACGCC ATCGACGCGG TGGTGGAGGA | 18150 |
| GGCGCGCAAC TACACGCCCA CGCCGCCACC AGTGTCCACA GTGGACGCGG | 18200 |
| CCATTCAGAC CGTGGTGCGC GGAGCCCGGC GCTATGCTAA AATGAAGAGA | 18250 |
| CGGCGGAGGC GCGTAGCACG TCGCCACCGC CGCCGACCCG GCACTGCCGC | 18300 |
| CCAACGCGCG GCGGCGGCCC TGCTTAACCG CGCACGTCGC ACCGGCCGAC | 18350 |
| GGGCGGCCAT GCGGGCCGCT CGAAGGCTGG CCGCGGGTAT TGTCACTGTG | 18400 |
| CCCCCCAGGT CCAGGCGACG AGCGGCCGCC GCAGCAGCCG CGGCCATTAG | 18450 |
| TGCTATGACT CAGGGTCGCA GGGGCAACGT GTATTGGGTG CGCGACTCGG | 18500 |
| TTAGCGGCCT GCGCGTGCCC GTGCGCACCC GCCCCCCGCG CAACTAGATT | 18550 |
| GCAAGAAAAA ACTACTTAGA CTCGTACTGT TGTATGTATC CAGCGGCGGC | 18600 |
| GGCGCGCAAC GAAGCTATGT CCAAGCGCAA AATCAAAGAA GAGATGCTCC | 18650 |
| AGGTCATCGC GCCGGAGATC TATGGCCCCC CGAAGAAGGA AGAGCAGGAT | 18700 |
| TACAAGCCCC GAAAGCTAAA GCGGGTCAAA AAGAAAAAGA AAGATGATGA | 18750 |
| TGATGAACTT GACGACGAGG TGGAACTGCT GCACGCTACC GCGCCCAGGC | 18800 |
| GACGGGTACA GTGGAAAGGT CGACGCGTAA AACGTGTTTT GCGACCCGGC | 18850 |
| ACCACCGTAG TCTTTACGCC CGGTGAGCGC TCCACCCGCA CCTACAAGCG | 18900 |
| CGTGTATGAT GAGGTGTACG GCGACGAGGA CCTGCTTGAG CAGGCCAACG | 18950 |
| AGCGCCTCGG GGAGTTTGCC TACGGAAAGC GGCATAAGGA CATGCTGGCG | 19000 |
| TTGCCGCTGG ACGAGGGCAA CCCAACACCT AGCCTAAAGC CCGTAACACT | 19050 |
| GCAGCAGGTG CTGCCCGCGC TTGCACCGTC CGAAGAAAAG CGCGGCCTAA | 19100 |
| AGCGCGAGTC TGGTGACTTG GCACCCACCG TGCAGCTGAT GGTACCCAAG | 19150 |
| CGCCAGCGAC TGGAAGATGT CTTGGAAAAA ATGACCGTGG AACCTGGGCT | 19200 |
| GGAGCCCGAG GTCCGCGTGC GGCCAATCAA GCAGGTGGCG CCGGGACTGG | 19250 |
| GCGTGCAGAC CGTGGACGTT CAGATACCCA CTACCAGTAG CACCAGTATT | 19300 |
| GCCACCGCCA CAGAGGGCAT GGAGACACAA ACGTCCCCGG TTGCCTCAGC | 19350 |
| GGTGGCGGAT GCCGCGGTGC AGGCGGTCGC TGCGGCCGCG TCCAAGACCT | 19400 |
| CTACGGAGGT GCAAACGGAC CCGTGGATGT TTCGCGTTTC AGCCCCCCGG | 19450 |
| CGCCCGCGCC GTTCGAGGAA GTACGGCGCC GCCAGCGCGC TACTGCCCGA | 19500 |
| ATATGCCCTA CATCCTTCCA TTGCGCCTAC CCCCGGCTAT CGTGGCTACA | 19550 |
| CCTACCGCCC CAGAAGACGA GCAACTACCC GACGCCGAAC CACCACTGGA | 19600 |
| ACCCGCCGCC GCCGTCGCCG TCGCCAGCCC GTGCTGGCCC CGATTTCCGT | 19650 |
| GCGCAGGGTG GCTCGCGAAG GAGGCAGGAC CCTGGTGCTG CCAACAGCGC | 19700 |
| GCTACCACCC CAGCATCGTT TAAAAGCCGG TCTTTGTGGT TCTTGCAGAT | 19750 |
| ATGGCCCTCA CCTGCCGCCT CCGTTTCCCG GTGCCGGGAT TCCGAGGAAG | 19800 |
| AATGCACCGT AGGAGGGGCA TGGCCGGCCA CGGCCTGACG GGCGGCATGC | 19850 |
| GTCGTGCGCA CCACCGGCGG CGGCGCGCGT CGCACCGTCG CATGCGCGGC | 19900 |
| GGTATCCTGC CCCTCCTTAT TCCACTGATC GCCGCGGCGA TTGGCGCCGT | 19950 |
| GCCCGGAATT GCATCCGTGG CCTTGCAGGC GCAGAGACAC TGATTAAAAA | 20000 |
| CAAGTTGCAT GTGGAAAAAT CAAAATAAAA AGTCTGGACT CTCACGCTCG | 20050 |
| CTTGGTCCTG TAACTATTTT GTAGAATGGA AGACATCAAC TTTGCGTCTC | 20100 |
| TGGCCCCGCG ACACGGCTCG CGCCCGTTCA TGGGAAACTG GCAAGATATC | 20150 |
| GGCACCAGCA ATATGAGCGG TGGCGCCTTC AGCTGGGGCT CGCTGTGGAG | 20200 |
| CGGCATTAAA AATTTCGGTT CCACCGTTAA GAACTATGGC AGCAAGGCCT | 20250 |
| GGAACAGCAG CACAGGCCAG ATGCTGAGGG ATAAGTTGAA AGAGCAAAAT | 20300 |
| TTCCAACAAA AGGTGGTAGA TGGCCTGGCC TCTGGCATTA GCGGGGTGGT | 20350 |
| GGACCTGGCC AACCAGGCAG TGCAAAATAA GATTAACAGT AAGCTTGATC | 20400 |
| CCCGCCCTCC CGTAGAGGAG CCTCCACCGG CCGTGGAGAC AGTGTCTCCA | 20450 |
| GAGGGGCGTG GCGAAAAGCG TCCGCGCCCC GACAGGGAAG AAACTCTGGT | 20500 |
| GACGCAAATA GACGAGCCTC CCTCGTACGA GGAGGCACTA AAGCAAGGCC | 20550 |
| TGCCCACCAC CCGTCCCATC GCGCCCATGG CTACCGGAGT GCTGGGCCAG | 20600 |
| CACACACCCG TAACGCTGGA CCTGCCTCCC CCCGCCGACA CCCAGCAGAA | 20650 |
| ACCTGTGCTG CCAGGCCCGA CCGCCGTIGT TGTAACCCGT CCTAGCCGCG | 20700 |
| CGTCCCTGCG CCGCGCCGCC AGCGGTCCGC GATCGTTGCG GCCCGTAGCC | 20750 |
| AGTGGCAACT GGCAAAGCAC ACTGAACAGC ATCGTGGGTC TGGGGGTGCA | 20800 |
| ATCCCTGAAG CGCCGACGAT GCTTCTGATA GCTAACGTGT CGTATGTGTG | 20850 |
| TCATGTATGC GTCCATGTCG CCGCCAGAGG AGCTGCTGAG CCGCCGCGCG | 20900 |
| CCCGCTTTCC AAGATGGCTA CCCCTTCGAT GATGCCGCAG TGGTCTTACA | 20950 |
| TGCACATCTC GGGCCAGGAC GCCTCGGAGT ACCTGAGCCC CGGGCTGGTG | 21000 |
| CAGTTTGCCC GCGCCACCGA GACGTACTIC AGCCTGAATA ACAAGTTTAG | 21050 |
| AAACCCCACG GTGGCGCCTA CGCACGACGT GACCACAGAC CGGTCCCAGC | 21100 |
| GTTTGACGCT GCGGTTCATC CCTGTGGACC GTGAGGATAC TGCGTACTCG | 21150 |
| TACAAGGCGC GGTTCACCCT AGCTGTGGGT GATAACCGTG TGCTGGACAT | 21200 |
| GGCTTCCACG TACTTTGACA TCCGCGGCGT GCTGGACAGG GGCCCTACTT | 21250 |
| TTAAGCCCTA CTCTGGCACT GCCTACAACG CCCTGGCTCC CAAGGGTGCC | 21300 |
| CCAAATCCTT GCGAATGGGA TGAAGCTGCT ACTGCTCTTG AAATAAACCT | 21350 |
| AGAAGAAGAG GACGATGACA ACGAAGACGA AGTAGACGAG CAAGCTGAGC | 21400 |
| AGCAAAAAAC TCACGTATTT GGGCAGGCGC CTTATTCTGG TATAAATATT | 21450 |
| ACAAAGGAGG GTATTCAAAT AGGTGTCGAA GGTCAAACAC CTAAATATGC | 21500 |
| CGATAAAACA TTTCAACCTG AACCTCAAAT AGGAGAATCT CAGTGGTACG | 21550 |
| AAACAGAAAT TAATCATGCA GCTGGGAGAG TCCTAAAAAA GACTACCCCA | 21600 |
| ATGAAACCAT GTTACGGTTC ATATGCAAAA CCCACAAATG AAAATGGAGG | 21650 |
| GCAAGGCATT CTTGTAAAGC AACAAAATGG AAAGCTAGAA AGTCAAGTGG | 21700 |
| AAATGCAATT TTTCTCAACT ACTGAGGCAG CCGCAGGCAA TGGTGATAAC | 21750 |
| TTGACTCCTA AAGTGGTATT GTACAGTGAA GATGTAGATA TAGAAACCCC | 21800 |
| AGACACTCAT ATTTCTTACA TGCCCACTAT TAAGGAAGGT AACTCACGAG | 21850 |
| AACTAATGGG CCAACAATCT ATGCCCAACA GGCCTAATTA CATTGCTTTT | 21900 |
| AGGGACAATT TTATTGGTCT AATGTATTAC AACAGCACGG GTAATATGGG | 21950 |
| TGTTCTGGCG GGCCAAGCAT CGCAGTTGAA TGCTGTTGTA GATTTGCAAG | 22000 |
| ACAGAAACAC AGAGCTTTCA TACCAGCTTT TGCTTGATTC CATTGGTGAT | 22050 |
| AGAACCAGGT ACTTTTCTAT GTGGAATCAG GCTGTTGACA GCTATGATCC | 22100 |
| AGATGTTAGA ATTATTGAAA ATCATGGAAC TGAAGATGAA CTTCCAAATT | 22150 |
| ACTGCTTTCC ACTGGGAGGT GTGATTAATA CAGAGACTCT TACCAAGGTA | 22200 |
| AAACCTAAAA CAGGTCAGGA AAATGGATGG GAAAAAGATG CTACAGAATT | 22250 |
| TTCAGATAAA AATGAAATAA GAGTTGGAAA TAATTTTGCC ATGGAAATCA | 22300 |
| ATCTAAATGC CAACCTGTGG AGAAATTTCC TGTACTCCAA CATAGCGCTG | 22350 |
| TATTTGCCCG ACAAGCTAAA GTACAGTCCT TCCAACGTAA AAATTTCTGA | 22400 |
| TAACCCAAAC ACCTACGACT ACATGAACAA GCGAGTGGTG GCTCCCGGGC | 22450 |
| TAGTGGACTG CTACATTAAC CTTGGAGCAC GCTGGTCCCT TGACTATATG | 22500 |
| GACAACGTCA ACCCATTTAA CCACCACCGC AATGCTGGCC TGCGCTACCG | 22550 |
| CTCAATGTTG CTGGGCAATG GTCGCTATGT GCCCTTCCAC ATCCAGGTGC | 22600 |
| CTCAGAAGTT CTTTGCCATT AAAAACCTCC TTCTCCTGCC GGGCTCATAC | 22650 |
| ACCTACGAGT GGAACTTCAG GAAGGATGTT AACATGGTTC TGCAGAGCTC | 22700 |
| CCTAGGAAAT GACCTAAGGG TTGACGGAGC CAGCATTAAG TTTGATAGCA | 22750 |
| TTTGCCTTTA CGCCACCTTC TTCCCCATGG CCCACAACAC CGCCTCCACG | 22800 |
| CTTGAGGCCA TGCTTAGAAA CGACACCAAC GACCAGTCCT TTAACGACTA | 22850 |
| TCTCTCCGCC GCCAACATGC TCTACCCTAT ACCCGCCAAC GCTACCAACG | 22900 |
| TGCCCATATC CATCCCCTCC CGCAACTGGG CGGCTTTCCG CGGCTGGGCC | 22950 |
| TTCACGCGCC TTAAGACTAA GGAAACCCCA TCACTGGGCT CGGGCTACGA | 23000 |
| CCCTTATTAC ACCTACTCTG GCTCTATACC CTACCTAGAT GGAACCTTTT | 23050 |
| ACCTCAACCA CACCTTTAAG AAGGTGGCCA TTACCTTTGA CTCTTCTGTC | 23100 |
| AGCTGGCCTG GCAATGACCG CCTGCTTACC CCCAACGAGT TTGAAATTAA | 23150 |
| GCGCTCAGTT GACGGGGAGG GTTACAACGT TGCCCAGTGT AACATGACCA | 23200 |
| AAGACTGGTT CCTGGTACAA ATGCTAGCTA ACTATAACAT TGGCTACCAG | 23250 |
| GGCTTCTATA TCCCAGAGAG CTACAAGGAC CGCATGTACT CCTTCTTTAG | 23300 |
| AAACTTCCAG CCCATGAGCC GTCAGGTGGT GGATGATACT AAATACAAGG | 23350 |
| ACTACCAACA GGTGGGCATC CTACACCAAC ACAACAACTC TGGATTTGTT | 23400 |
| GGCTACCTTG CCCCCACCAT GCGCGAAGGA CAGGCCTACC CTGCTAACTT | 23450 |
| CCCCTATCCG CTTATAGGCA AGACCGCAGT TGACAGCATT ACCCAGAAAA | 23500 |
| AGTTTCTTTG CGATCGCACC CTTTGGCGCA TCCCATTCTC CAGTAACTTT | 23550 |
| ATGTCCATGG GCGCACTCAC AGACCTGGGC CAAAACCTTC TCTACGCCAA | 23600 |
| CTCCGCCCAC GCGCTAGACA TGACTTTTGA GGTGGATCCC ATGGACGAGC | 23650 |
| CCACCCTTCT TTATGTTTTG TTTGAAGTCT TTGACGTGGT CCGTGTGCAC | 23700 |
| CAGCCGCACC GCGGCGTCAT CGAAACCGTG TACCTGCGCA CGCCCTTCTC | 23750 |
| GGCCGGCAAC GCCACAACAT AAAGAAGCAA GCAACATCAA CAACAGCTGC | 23800 |
| CGCCATGGGC TCCAGTGAGC AGGAACTGAA AGCCATTGTC AAAGATCTTG | 23850 |
| GTTGTGGGCC ATATTTTTTG GGCACCTATG ACAAGCGCTT TCCAGGCTTT | 23900 |
| GTTTCTCCAC ACAAGCTCGC CTGCGCCATA GTCAATACGG CCGGTCGCGA | 23950 |
| GACTGGGGGC GTACACTGGA TGGCCTTTGC CTGGAACCCG CACTCAAAAA | 24000 |
| CATGCTACCT CTTTGAGCCC TTTGGCTTTT CTGACCAGCG ACTCAAGCAG | 24050 |
| GTTTACCAGT TTGAGTACGA GTCACTCCTG CGCCGTAGCG CCATTGCTTC | 24100 |
| TTCCCCCGAC CGCTGTATAA CGCTGGAAAA GTCCACCCAA AGCGTACAGG | 24150 |
| GGCCCAACTC GGCCGCCTGT GGACTATTCT GCTGCATGTT TCTCCACGCC | 24200 |
| TTTGCCAACT GGCCCCAAAC TCCCATGGAT CACAACCCCA CCATGAACCT | 24250 |
| TATTACCGGG GTACCCAACT CCATGCTCAA CAGTCCCCAG GTACAGCCCA | 24300 |
| CCCTGCGTCG CAACCAGGAA CAGCTCTACA GCTTCCTGGA GCGCCACTCG | 24350 |
| CCCTACTTCC GCAGCCACAG TGCGCAGATT AGGAGCGCCA CTTCTTTTTG | 24400 |
| TCACTTGAAA AACATGTAAA AATAATGTAC TAGAGACACT TTCAATAAAG | 24450 |
| GCAAATGCTT TTATTTGTAC ACTCTCGGGT GATTATTTAC CCCCACCCTT | 24500 |
| GCCGTCTGCG CCGTTTAAAA ATCAAAGGGG TTCTGCCGCG CATCGCTATG | 24550 |
| CGCCACTGGC AGGGACACGT TGCGATACTG GTGTTTAGTG CTCCACTTAA | 24600 |
| ACTCAGGCAC AACCATCCGC GGCAGCTCGG TGAAGTTTTC ACTCCACAGG | 24650 |
| CTGCGCACCA TCACCAACGC GTTTAGCAGG TCGGGCGCCG ATATCTTGAA | 24700 |
| GTCGCAGTTG GGGCCTCCGC CCTGCGCGCG CGAGTTGCGA TACACAGGGT | 24750 |
| TGCAGCACTG GAACACTATC AGCGCCGGGT GGTGCACGCT GGCCAGCACG | 24800 |
| CTCTTGTCGG AGATCAGATC CGCGTCCAGG TCCTCCGCGT TGCTCAGGGC | 24850 |
| GAACGGAGTC AACTTTGGTA GCTGCCTTCC CAAAAAGGGC GCGTGCCCAG | 24900 |
| GCTTTGAGTT GCACTCGCAC CGTAGTGGCA TCAAAAGGTG ACCGTGCCCG | 24950 |
| GTCTGGGCGT TAGGATACAG CGCCTGCATA AAAGCCTTGA TCTGCTTAAA | 25000 |
| AGCCACCTGA GCCTTTGCGC CTTCAGAGAA GAACATGCCG CAAGACTTGC | 25050 |
| CGGAAAACTG ATTGGCCGGA CAGGCCGCGT CGTGCACGCA GCACCTTGCG | 25100 |
| TCGGTGTTGG AGATCTGCAC CACATTTCGG CCCCACCGGT TCTTCACGAT | 25150 |
| CTTGGCCTTG CTAGACTGCT CCTTCAGCGC GCGCTGCCCG TTTTCGCTCG | 25200 |
| TCACATCCAT TTCAATCACG TGCTCCTTAT TTATCATAAT GCTTCCGTGT | 25250 |
| AGACACTTAA GCTCGCCTTC GATCTCAGCG CAGCGGTGCA GCCACAACGC | 25300 |
| GCAGCCCGTG GGCTCGTGAT GCTTGTAGGT CACCTCTGCA AACGACTGCA | 25350 |
| GGTACGCCTG CAGGAATCGC CCCATCATCG TCACAAAGGT CTTGTTGCTG | 25400 |
| GTGAAGGICA GCTGCAACCC GCGGTGCTCC TCGTTCAGCC AGGTCTTGCA | 25450 |
| TACGGCCGCC AGAGCTTCCA CTTGGTCAGG CAGTAGTTTG AAGTTCGCCT | 25500 |
| TTAGATCGTT ATCCACGTGG TACTTGTCCA TCAGCGCGCG CGCAGCCTCC | 25550 |
| ATGCCCTTCT CCCACGCAGA CACGATCGGC ACACTCAGCG GGTTCATCAC | 25600 |
| CGTAATTTCA CTTTCCGCTT CGCTGGGCTC TTCCTCTTCC TCTTGCGTCC | 25650 |
| GCATACCACG CGCCACTGGG TCGTCTTCAT TCAGCCGCCG CACTGTGCGC | 25700 |
| TTACCTCCTT TGCCATGCTT GATTAGCACC GGTGGGTTGC TGAAACCCAC | 25750 |
| CATTTGTAGC GCCACATCTT CTCTTTCTTC CTCGCTGTCC ACGATTACCT | 25800 |
| CTGGTGATGG CGGGCGCTCG GGCTTGGGAG AAGGGCGCTT CTTTTTCTTC | 25850 |
| TTGGGCGCAA TGGCCAAATC CGCCGCCGAG GTCGATGGCC GCGGGCTGGG | 25900 |
| TGTGCGCGGC ACCAGCGCGT CTTGTGATGA GTCTTCCTCG TCCTCGGACT | 25950 |
| CGATACGCCG CCTCATCCGC TTTTTTGGGG GCGCCCGGGG AGGCGGCGGC | 26000 |
| GACGGGGACG GGGACGACAC GTCCTCCATG GTTGGGGGAC GTCGCGCCGC | 26050 |
| ACCGCGTCCG CGCTCGGGGG TGGTTTCGCG CTGCTCCTCT TCCCGACTGG | 26100 |
| CCATTTCCTT CTCCTATAGG CAGAAAAAGA TCATGGAGTC AGTCGAGAAG | 26150 |
| AAGGACAGCC TAACCGCCCC CTCTGAGTTC GCCACCACCG CCTCCACCGA | 26200 |
| TGCCGCCAAC GCGCCTACCA CCTTCCCCGT CGAGGCACCC CCGCTTGAGG | 26250 |
| AGGAGGAAGT GATTATCGAG CAGGACCCAG GTTTTGTAAG CGAAGACGAC | 26300 |
| GAGGACCGCT CAGTACCAAC AGAGGATAAA AAGCAAGACC AGGACAACGC | 26350 |
| AGAGGCAAAC GAGGAACAAG TCGGGCGGGG GGACGAAAGG CATGGCGACT | 26400 |
| ACCTAGATGT GGGAGACGAC GTGCTGTTGA AGCATCTGCA GCGCCAGTGC | 26450 |
| GCCATTATCT GCGACGCGTT GCAAGAGCGC AGCGATGTGC CCCTCGCCAT | 26500 |
| AGCGGATGTC AGCCTTGCCT ACGAACGCCA CCTATTCTCA CCGCGCGTAC | 26550 |
| CCCCCAAACG CCAAGAAAAC GGCACATGCG AGCCCAACCC GCGCCTCAAC | 26600 |
| TTCTACCCCG TATTTGCCGT GCCAGAGGTG CTTGCCACCT ATCACATCTT | 26650 |
| TTTCCAAAAC TGCAAGATAC CCCTATCCTG CCGTGCCAAC CGCAGCCGAG | 26700 |
| CGGACAAGCA GCTGGCCTTG CGGCAGGGCG CTGTCATACC TGATATCGCC | 26750 |
| TCGCTCAACG AAGTGCCAAA AATCTTTGAG GGTCTTGGAC GCGACGAGAA | 26800 |
| GCGCGCGGCA AACGCTCTGC AACAGGAAAA CAGCGAAAAT GAAAGTCACT | 26850 |
| CTGGAGTGTT GGTGGAACTC GAGGGTGACA ACGCGCGCCT AGCCGTACTA | 26900 |
| AAACGCAGCA TCGAGGTCAC CCACTTTGCC TACCCGGCAC TTAACCTACC | 26950 |
| CCCCAAGGTC ATGAGCACAG TCATGAGTGA GCTGATCGTG CGCCGTGCGC | 27000 |
| AGCCCCTGGA GAGGGATGCA AATTTGCAAG AACAAACAGA GGAGGGCCTA | 27050 |
| CCCGCAGTTG GCGACGAGCA GCTAGCGCGC TGGCTTCAAA CGCGCGAGCC | 27100 |
| TGCCGACTTG GAGGAGCGAC GCAAACTAAT GATGGCCGCA GTGCTCGTTA | 27150 |
| CCGTGGAGCT TGAGTGCATG CAGCGGTTCT TTGCTGACCC GGAGATGCAG | 27200 |
| CGCAAGCTAG AGGAAACATT GCACTACACC TTTCGACAGG GCTACGTACG | 27250 |
| CCAGGCCTGC AAGATCTCCA ACGTGGAGCT CTGCAACCTG GTCTCCTACC | 27300 |
| TTGGAATTTT GCACGAAAAC CGCCTTGGGC AAAACGTGCT TCATTCCACG | 27350 |
| CTCAAGGGCG AGGCGCGCCG CGACTACGTC CGCGACTGCG TTTACTTATT | 27400 |
| TCTATGCTAC ACCTGGCAGA CGGCCATGGG CGTTTGGCAG CAGTGCTTGG | 27450 |
| AGGAGTGCAA CCTCAAGGAG CTGCAGAAAC TGCTAAAGCA AAACTTGAAG | 27500 |
| GACCTATGGA CGGCCTTCAA CGAGCGCTCC GTGGCCGCGC ACCTGGCGGA | 27550 |
| CATCATTTTC CCCGAACGCC TGCTTAAAAC CCTGCAACAG GGTCTGCCAG | 27600 |
| ACTTCACCAG TCAAAGCATG TTGCAGAACT TTAGGAACTT TATCCTAGAG | 27650 |
| CGCTCAGGAA TCTTGCCCGC CACCTGCTGT GCACTTCCTA GCGACTTTGT | 27700 |
| GCCCATTAAG TACCGCGAAT GCCCTCCGCC GCTTTGGGGC CACTGCTACC | 27750 |
| TTCTGCAGCT AGCCAACTAC CTTGCCTACC ACTCTGACAT AATGGAAGAC | 27800 |
| GTGAGCGGTG ACGGTCTACT GGAGTGTCAC TGTCGCTGCA ACCTATGCAC | 27850 |
| CCCGCACCGC TCCCTGGTTT GCAATTCGCA GCTGCTTAAC GAAAGTCAAA | 27900 |
| TTATCGGTAC CTTTGAGCTG CAGGGTCCCT CGCCTGACGA AAAGTCCGCG | 27950 |
| GCTCCGGGGT TGAAACTCAC TCCGGGGCTG TGGACGTCGG CTTACCTTCG | 28000 |
| CAAATTTGTA CCTGAGGACT ACCACGCCCA CGAGATTAGG TTCTACGAAG | 28050 |
| ACCAATCCCG CCCGCCTAAT GCGGAGCTTA CCGCCTGCGT CATTACCCAG | 28100 |
| GGCCACATTC TTGGCCAATT GCAAGCCATC AACAAAGCCC GCCAAGAGTT | 28150 |
| TCTGCTACGA AAGGGACGGG GGGTTTACTT GGACCCCCAG TCCGGCGAGG | 28200 |
| AGCTCAACCC AATCCCCCCG CCGCCGCAGC CCTATCAGCA GCAGCCGCGG | 28250 |
| GCCCTTGCTT CCCAGGATGG CACCCAAAAA GAAGCTGCAG CTGCCGCCGC | 28300 |
| CACCCACGGA CGAGGAGGAA TACTGGGACA GTCAGGCAGA GGAGGTTTTG | 28350 |
| GACGAGGAGG AGGAGGACAT GATGGAAGAC TGGGAGAGCC TAGACGAGGA | 28400 |
| AGCTTCCGAG GTCGAAGAGG TGTCAGACGA AACACCGTCA CCCTCGGTCG | 28450 |
| CATTCCCCTC GCCGGCGCCC CAGAAATCGG CAACCGGTTC CAGCATGGCT | 28500 |
| ACAACCTCCG CTCCTCAGGC GCCGCCGGCA CTGCCCGTTC GCCGACCCAA | 28550 |
| CCGTAGATGG GACACCACTG GAACCAGGGC CGGTAAGTCC AAGCAGCCGC | 28600 |
| CGCCGTTAGC CCAAGAGCAA CAACAGCGCC AAGGCTACCG CTCATGGCGC | 28650 |
| GGGCACAAGA ACGCCATAGT TGCTTGCTTG CAAGACTGTG GGGGCAACAT | 28700 |
| CTCCTTCGCC CGCCGCTTTC TTCTCTACCA TCACGGCGTG GCCTTCCCCC | 28750 |
| GTAACATCCT GCATTACTAC CGTCATCTCT ACAGCCCATA CTGCACCGGC | 28800 |
| GGCAGCGGCA GCAACAGCAG CGGCCACACA GAAGCAAAGG CGACCGGATA | 28850 |
| GCAAGACTCT GACAAAGCCC AAGAAATCCA CAGCGGCGGC AGCAGCAGGA | 28900 |
| GGAGGAGCGC TGCGTCTGGC GCCCAACGAA CCCGTATCGA CCCGCGAGCT | 28950 |
| TAGAAACAGG ATTTTTCCCA CTCTGTATGC TATATTICAA CAGAGCAGGG | 29000 |
| GCCAAGAACA AGAGCTGAAA ATAAAAAACA GGTCTCTGCG ATCCCTCACC | 29050 |
| CGCAGCTGCC TGTATCACAA AAGCGAAGAT CAGCTTCGGC GCACGCTGGA | 29100 |
| AGACGCGGAG GCTCTCTTCA GTAAATACTG CGCGCTGACT CTTAAGGACT | 29150 |
| AGTTTCGCGC CCTTTCTCAA ATTTAAGCGC GAAAACTACG TCATCTCCAG | 29200 |
| CGGCCACACC CGGCGCCAGC ACCTGTTGTC AGCGCCATTA TGAGCAAGGA | 29250 |
| AATTCCCACG CCCTACATGT GGAGTTACCA GCCACAAATG GGACTTGCGG | 29300 |
| CTGGAGCTGC CCAAGACTAC TCAACCCGAA TAAACTACAT GAGCGCGGGA | 29350 |
| CCCCACATGA TATCCCGGGT CAACGGAATA CGCGCCCACC GAAACCGAAT | 29400 |
| TCTCCTGGAA CAGGCGGCTA TTACCACCAC ACCTCGTAAT AACCTTAATC | 29450 |
| CCCGTAGTTG GCCCGCTGCC CTGGTGTACC AGGAAAGTCC CGCTCCCACC | 29500 |
| ACTGTGGTAC TTCCCAGAGA CGCCCAGGCC GAAGTTCAGA TGACTAACTC | 29550 |
| AGGGGCGCAG CTTGCGGGCG GCTTTCGTCA CAGGGTGCGG TCGCCCGGGC | 29600 |
| AGGGTATAAC TCACCTGACA ATCAGAGGGC GAGGTATTCA GCTCAACGAC | 29650 |
| GAGTCGGTGA GCTCCTCGCT TGGTCTCCGT CCGGACGGGA CATTTCAGAT | 29700 |
| CGGCGGCGCC GGCCGCTCTT CATTCACGCC TCGTCAGGCA ATCCTAACTC | 29750 |
| TGCAGACCTC GTCCTCTGAG CCGCGCTCTG GAGGCATTGG AACTCTGCAA | 29800 |
| TTTATTGAGG AGTTTGTGCC ATCGGTCTAC TTTAACCCCT TCTCGGGACC | 29850 |
| TCCCGGCCAC TATCCGGATC AATTTATTCC TAACTTTGAC GCGGTAAAGG | 29900 |
| ACTCGGCGGA CGGCTACGAC TGAATGTTAA GTGGAGAGGC AGAGCAACTG | 29950 |
| CGCCTGAAAC ACCTGGTCCA CTGTCGCCGC CACAAGTGCT TTGCCCGCGA | 30000 |
| CTCCGGTGAG TTTTGCTACT TTGAATTGCC CGAGGATCAT ATCGAGGGCC | 30050 |
| CGGCGCACGG CGTCCGGCTT ACCGCCCAGG GAGAGCTTGC CCGTAGCCTG | 30100 |
| ATTCGGGAGT TTACCCAGCG CCCCCTGCTA GTTGAGCGGG ACAGGGGACC | 30150 |
| CTGTGTTCTC ACTGTGATTT GCAACTGTCC TAACCCTGGA TTACATCAAG | 30200 |
| ATCCTCTAGT TAATGTCAGG TCGCCTAAGT CGATTAACTA GAGTACCCGG | 30250 |
| GGATCTTATT CCCTTTAACT AATAAAAAAA AATAATAAAG CATCACTTAC | 30300 |
| TTAAAATCAG TTAGCAAATT TCTGTCCAGT TTATTCAGCA GCACCTCCTT | 30350 |
| GCCCTCCTCC CAGCTCTGGT ATTGCAGCTT CCTCCTGGCT GCAAACTTTC | 30400 |
| TCCACAATCT AAATGGAATG TCAGTTTCCT CCTGTTCCTG TCCATCCGCA | 30450 |
| CCCACTATCT TCATGTTGTT GCAGATGAAG CGCGCAAGAC CGTCTGAAGA | 30500 |
| TACCTTCAAC CCCGTGTATC CATATGACAC GGAAACCGGT CCTCCAACTG | 30550 |
| TGCCTTTTCT TACTCCTCCC TTTGTATCCC CCAATGGGTT TCAAGAGAGT | 30600 |
| CCCCCTGGGG TACTCTCTTT GCGCCTATCC GAACCTCTAG TTACCTCCAA | 30650 |
| TGGCATGCTT GCGCTCAAAA TGGGCAACGG CCTCTCTCTG GACGAGGCCG | 30700 |
| GCAACCTTAC CTCCCAAAAT GTAACCACTG TGAGCCCACC TCTCAAAAAA | 30750 |
| ACCAAGTCAA ACATAAACCT GGAAATATCT GCACCCCTCA CAGTTACCTC | 30800 |
| AGAAGCCCTA ACTGTGGCTG CCGCCGCACC TCTAATGGTC GCGGGCAACA | 30850 |
| CACTCACCAT GCAATCACAG GCCCCGCTAA CCGTGCACGA CTCCAAACTT | 30900 |
| AGCATTGCCA CCCAAGGACC CCTCACAGTG TCAGAAGGAA AGCTAGCCCT | 30950 |
| GCAAACATCA GGCCCCCTCA CCACCACCGA TAGCAGTACC CTTACTATCA | 31000 |
| CTGCCTCACC CCCTCTAACT ACTGCCACTG GTAGCTTGGG CATTGACTTG | 31050 |
| AAAGAGCCCA TTTATACACA AAATGGAAAA CTAGGACTAA AGTACGGGGC | 31100 |
| TCCTTTGCAT GTAACAGACG ACCTAAACAC TTTGACCGTA GCAACTGGTC | 31150 |
| CAGGTGTGAC TATTAATAAT ACTTCCTTGC AAACTAAAGT TACTGGAGCC | 31200 |
| TTGGGTTTTG ATTCACAAGG CAATATGCAA CTTAATGTAG CAGGAGGACT | 31250 |
| AAGGATTGAT TCTCAAAACA GACGCCTTAT ACTTGATGTT AGTTATCCGT | 31300 |
| TTGATGCTCA AAACCAACTA AATCTAAGAC TAGGACAGGG CCCTCTTTTT | 31350 |
| ATAAACTCAG CCCACAACTT GGATATTAAC TACAACAAAG GCCTTTACTT | 31400 |
| GTTTACAGCT TCAAACAATT CCAAAAAGCT TGAGGTTAAC CTAAGCACTG | 31450 |
| CCAAGGGGTT GATGTTTGAC GCTACAGCCA TAGCCATTAA TGCAGGAGAT | 31500 |
| GGGCTTGAAT TTGGTTCACC TAATGCACCA AACACAAATC CCCTCAAAAC | 31550 |
| AAAAATTGGC CATGGCCTAG AATTTGATTC AAACAAGGCT ATGGTTCCTA | 31600 |
| AACTAGGAAC TGGCCTTAGT TTTGACAGCA CAGGTGCCAT TACAGTAGGA | 31650 |
| AACAAAAATA ATGATAAGCT AACTTTGTGG ACCACACCAG CTCCATCTCC | 31700 |
| TAACTGTAGA CTAAATGCAG AGAAAGATGC TAAACTCACT TTGGTCTTAA | 31750 |
| CAAAATGTGG CAGTCAAATA CTTGCTACAG TTTCAGTTTT GGCTGTTAAA | 31800 |
| GGCAGTTTGG CTCCAATATC TGGAACAGTT CAAAGTGCTC ATCTTATTAT | 31850 |
| AAGATTTGAC GAAAATGGAG TGCTACTAAA CAATTCCTTC CTGGACCCAG | 31900 |
| AATATTGGAA CTTTAGAAAT GGAGATCTTA CTGAAGGCAC AGCCTATACA | 31950 |
| AACGCTGTTG GATTTATGCC TAACCTATCA GCTTATCCAA AATCTCACGG | 32000 |
| TAAAACTGCC AAAAGTAACA TTGTCAGTCA AGTTTACTTA AACGGAGACA | 32050 |
| AAACTAAACC TGTAACACTA ACCATTACAC TAAACGGTAC ACAGGAAACA | 32100 |
| GGAGACACAA CTCCAAGTGC ATACTCTATG TCATTTTCAT GGGACTGGTC | 32150 |
| TGGCCACAAC TACATTAATG AAATATTTGC CACATCCTCT TACACTTTTT | 32200 |
| CATACATTGC CCAAGAATAA AGAATCGTTT GTGTTATGTT TCAACGTGTT | 32250 |
| TATTTTTCAA TTGCAGAAAA TTTCAAGTCA TTTTTCATTC AGTAGTATAG | 32300 |
| CCCCACCACC ACATAGCTTA TACAGATCAC CGTACCTTAA TCAAACTCAC | 32350 |
| AGAACCCTAG TATTCAACCT GCCACCTCCC TCCCAACACA CAGAGTACAC | 32400 |
| AGTCCTTTCT CCCCGGCTGG CCTTAAAAAG CATCATATCA TGGGTAACAG | 32450 |
| ACATATTCTT AGGTGTTATA TTCCACACGG TTTCCTGTCG AGCCAAACGC | 32500 |
| TCATCAGIGA TATTAATAAA CTCCCCGGGC AGCTCACTTA AGTTCATGTC | 32550 |
| GCTGTCCAGC TGCTGAGCCA CAGGCTGCTG TCCAACTTGC GGTTGCTTAA | 32600 |
| CGGGCGGCGA AGGAGAAGTC CACGCCTACA TGGGGGTAGA GTCATAATCG | 32650 |
| TGCATCAGGA TAGGGCGGTG GTGCTGCAGC AGCGCGCGAA TAAACTGCTG | 32700 |
| CCGCCGCCGC TCCGTCCTGC AGGAATACAA CATGGCAGTG GTCTCCTCAG | 32750 |
| CGATGATTCG CACCGCCCGC AGCATAAGGC GCCTTGTCCT CCGGGCACAG | 32800 |
| CAGCGCACCC TGATCTCACT TAAATCAGCA CAGTAACTGC AGCACAGCAC | 32850 |
| CACAATATTG TTCAAAATCC CACAGTGCAA GGCGCTGTAT CCAAAGCTCA | 32900 |
| TGGCGGGGAC CACAGAACCC ACGTGGCCAT CATACCACAA GCGCAGGTAG | 32950 |
| ATTAAGTGGC GACCCCTCAT AAACACGCTG GACATAAACA TTACCTCTTT | 33000 |
| TGGCATGTTG TAATTCACCA CCTCCCGGTA CCATATAAAC CTCTGATTAA | 33050 |
| ACATGGCGCC ATCCACCACC ATCCTAAACC AGCTGGCCAA AACCTGCCCG | 33100 |
| CCGGCTATAC ACTGCAGGGA ACCGGGACTG GAACAATGAC AGTGGAGAGC | 33150 |
| CCAGGACTCG TAACCATGGA TCATCATGCT CGTCATGATA TCAATGTTGG | 33200 |
| CACAACACAG GCACACGTGC ATACACTTCC TCAGGATTAC AAGCTCCTCC | 33250 |
| CGCGTTAGAA CCATATCCCA GGGAACAACC CATTCCTGAA TCAGCGTAAA | 33300 |
| TCCCACACTG CAGGGAAGAC CTCGCACGTA ACTCACGTTG TGCATTGTCA | 33350 |
| AAGTGTTACA TTCGGGCAGC AGCGGATGAT CCTCCAGTAT GGTAGCGCGG | 33400 |
| GTTTCTGTCT CAAAAGGAGG TAGACGATCC CTACTGTACG GAGTGCGCCG | 33450 |
| AGACAACCGA GATCGTGTTG GTCGTAGTGT CATGCCAAAT GGAACGCCGG | 33500 |
| ACGTAGTCAT ATTTCCTGAA GCAAAACCAG GTGCGGGCGT GACAAACAGA | 33550 |
| TCTGCGTCTC CGGTCTCGCC GCTTAGATCG CTCTGTGTAG TAGTTGTAGT | 33600 |
| ATATCCACTC TCTCAAAGCA TCCAGGCGCC CCCTGGCTTC GGGTTCTATG | 33650 |
| TAAACTCCTT CATGCGCCGC TGCCCTGATA ACATCCACCA CCGCAGAATA | 33700 |
| AGCCACACCC AGCCAACCTA CACATTCGTT CTGCGAGTCA CACACGGGAG | 33750 |
| GAGCGGGAAG AGCTGGAAGA ACCATGTTTT TTTTTTTATT CCAAAAGATT | 33800 |
| ATCCAAAACC TCAAAATGAA GATCTATTAA GTGAACGCGC TCCCCTCCGG | 33850 |
| TGGCGTGGTC AAACTCTACA GCCAAAGAAC AGATAATGGC ATTTGTAAGA | 33900 |
| TGTTGCACAA TGGCTTCCAA AAGGCAAACG GCCCTCACGT CCAAGTGGAC | 33950 |
| GTAAAGGCTA AACCCTTCAG GGTGAATCTC CTCTATAAAC ATTCCAGCAC | 34000 |
| CTTCAACCAT GCCCAAATAA TTCTCATCTC GCCACCTTCT CAATATATCT | 34050 |
| CTAAGCAAAT CCCGAATATT AAGTCCGGCC ATTGTAAAAA TCTGCTCCAG | 34100 |
| AGCGCCCTCC ACCTTCAGCC TCAAGCAGCG AATCATGATT GCAAAAATTC | 34150 |
| AGGTTCCICA CAGACCTGTA TAAGATTCAA AAGCGGAACA TTAACAAAAA | 34200 |
| TACCGCGATC CCGTAGGTCC CTTCGCAGGG CCAGCTGAAC ATAATCGTGC | 34250 |
| AGGTCTGCAC GGACCAGCGC GGCCACTTCC CCGCCAGGAA CCATGACAAA | 34300 |
| AGAACCCACA CTGATTATGA CACGCATACT CGGAGCTATG CTAACCAGCG | 34350 |
| TAGCCCCGAT GTAAGCTTGT TGCATGGGCG GCGATATAAA ATGCAAGGTG | 34400 |
| CTGCTCAAAA AATCAGGCAA AGCCTCGCGC AAAAAAGAAA GCACATCGTA | 34450 |
| GTCATGCTCA TGCAGATAAA GGCAGGTAAG CTCCGGAACC ACCACAGAAA | 34500 |
| AAGACACCAT TTTTCTCTCA AACATGTCTG CGGGTTTCTG CATAAACACA | 34550 |
| AAATAAAATA ACAAAAAAAC ATTTAAACAT TAGAAGCCTG TCTTACAACA | 34600 |
| GGAAAAACAA CCCTTATAAG CATAAGACGG ACTACGGCCA TGCCGGCGTG | 34650 |
| ACCGTAAAAA AACTGGTCAC CGTGATTAAA AAGCACCACC GACAGCTCCT | 34700 |
| CGGTCATGTC CGGAGTCATA ATGTAAGACT CGGTAAACAC ATCAGGTTGA | 34750 |
| TTCACATCGG TCAGTGCTAA AAAGCGACCG AAATAGCCCG GGGGAATACA | 34800 |
| TACCCGCAGG CGTAGAGACA ACATTACAGC CCCCATAGGA GGTATAACAA | 34850 |
| AATTAATAGG AGAGAAAAAC ACATAAACAC CTGAAAAACC CTCCTGCCTA | 34900 |
| GGCAAAATAG CACCCTCCCG CTCCAGAACA ACATACAGCG CTTCCACAGC | 34950 |
| GGCAGCCATA ACAGTCAGCC TTACCAGTAA AAAAGAAAAC CTATTAAAAA | 35000 |
| AACACCACTC GACACGGCAC CAGCTCAATC AGTCACAGTG TAAAAAAGGG | 35050 |
| CCAAGTGCAG AGCGAGTATA TATAGGACTA AAAAATGACG TAACGGTTAA | 35100 |
| AGTCCACAAA AAACACCCAG AAAACCGCAC GCGAACCTAC GCCCAGAAAC | 35150 |
| GAAAGCCAAA AAACCCACAA CTTCCTCAAA TCGTCACTTC CGTTTTCCCA | 35200 |
| CGTTACGTCA CTTCCCATTT TAAGAAAACT ACAATTCCCA ACACATACAA | 35250 |
| GTTACTCCGC CCTAAAACCT ACGTCACCCG CCCCGTTCCC ACGCCCCGCG | 35300 |
| CCACGTCACA AACTCCACCC CCTCATTATC ATATTGGCTT CAATCCAAAA | 35350 |
| TAAGGTATAT TATTGATGAT G | 35371 |

### Methods of Administration

In various embodiments, the viral vector is administered by a series of injections during TECAP. In various embodiments, the procedure comprises a series of intramyocardial injections to the left ventricle of the subject's heart. In various embodiments, the viral vector is administered through a series of 15 injections at separate delivery sites in the heart of the subject, wherein the viral vector diffuse through substantially all of the heart. In various embodiments each injection has an injection volume of about 0.1 mL. In various embodiments, the heart of the subject may be visualized throughout the procedure using a thorascope. As described in more detail below, use of a thorascope to visualize the subject's heart allows the administration of the viral vector through a minimally invasive procedure. In various embodiments, a dose of viral vector between about 1×10⁹ vp and about 1×10¹¹ vp is administered. In various embodiments, a dose of the viral vector of about 1×10⁹ vp, about 1×10¹⁰ vp, about 4×10¹⁰ vp or about 1×10¹¹ vp is administered. In various embodiments, the TECAP comprises making a 4-5 cm anterolateral incision in the 5th to 7th intercostal space of the subject. In various embodiments, the injections are made in the left ventricle.

### EXPERIMENTAL EXAMPLE

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in practicing the following examples are here described:

### Example 1

One strategy to prevent the consequences of atherosclerosis is to induce the existing blood vessels in the heart to create networks of new blood vessels to bypass the arterial system occluded by the atherosclerotic process, thus providing circulation to deliver sufficient oxygen needed by the tissue. The de novo creation of blood vessels, a process termed “angiogenesis,” is a complex, normal physiologic process that includes the regulated proliferation and migration of endothelial cells, localized dissolution of the basement membrane/extracellular matrix at the site of the sprouting neovessel, the migration of endothelial cells and their coalescence into tube-like structures, the reformation of the surrounding basement membrane, and the formation of the new vessels into networks with linkage to an appropriate venous system. The physiologic process of angiogenesis involves several mediators that function to produce new vessels in an ordered fashion. VEGF is one of the key components that initiates this process.

Myocardial administration of a gene coding for VEGF is a strategy using the delivery of genetic information to the myocardium to create networks of new blood vessels.

The most direct method of transferring genes to the myocardium is by injection under direct vision. This can be accomplished by exposure of the myocardium through a thoracoscopy, left thoracotomy or sternotomy, or by minimally invasive surgery. The advantages of a direct injection strategy are the following: (1) compared with other delivery techniques, the highest levels of localized transgene expression can be achieved, (2) vectors can be delivered with a high degree of accuracy, (3) a number of targeted injections can be performed and (4) limited systemic spread of the vector occurs.

Subjects in this study will undergo direct administration of XC001 expressing the human VEGF to induce therapeutic angiogenesis (revascularization). Access to the myocardium will be obtained via Transthoracic EpiCArdial Procedure or TECAP, a minimally invasive surgical approach for transthoracic epicardial access. VEGF is not only essential to the process of angiogenesis but, because it can be secreted from intact cells, it is ideal for gene transfer therapy aimed at improving perfusion to ischemic myocardium. Several clinical trials based on intramyocardial injection of VEGF DNA (as plasmids or expressed by adenovirus gene transfer vectors) in subjects with clinically significant CAD have been completed. These trials have documented the tolerability of gene transfer using plasmid DNA or adenovirus vectors coding for VEGF and show promise of, but have not proven, enhanced myocardial perfusion and reduced anginal symptoms in the treated subjects. Our proposed study, in contrast, will use a human VEGF cDNA/genomic hybrid that generates multiple naturally occurring VEGF isoforms, with a predominance of the heparin-binding isoforms that are more effectively retained locally which in animal studies appears to lead to an improvement in angiogenic potency.

### VEGF Gene Therapy

Therapeutic angiogenesis mediated through a vector-delivered genetic message for an angiogenic factor has been studied in animal models and in clinical trials since the late 1990s. Studies have included genes for many protein angiogenic factors delivered by plasmid as well as by viral vectors (particularly adenovirus) and a variety of other administrative routes and delivery systems. In non-malignant tissues, the human VEGF gene is expressed in multiple isoforms, secondary to post-transcriptional splicing. The VEGF protein is capable of inducing angiogenesis, however, delivery of VEGF protein for therapeutic purposes has presented a significant challenge because the half-life of VEGF is very short, administration of high doses of VEGF is associated with hypotension and edema, and systemic administration of VEGF carries the theoretical risk of promiscuous induction of angiogenesis in tissues other than the target organ. To circumvent these problems, the VEGF cDNA coding sequence can be used as the source of local VEGF at the site of administration. Optimization of XC001 to improve angiogenic potency following intramyocardial injection has involved use of a multiple-isoform approach which nonclinical studies indicate may yield even better clinical efficacy than did the AdVEGF121 precursor viral transfer agent and a construct to increase the ratio of heparin-binding isoforms expressed which may be expected to have a stronger local angiogenic effect due to their ability to more tightly bind to the extracellular matrix.

### Nonclinical Data

Angiogenic responses, including collateral vessel development with improvement in both myocardial perfusion and function has been demonstrated by the delivery of VEGF isoforms with adenovirus in swine heart and mouse hindlimb models. AdVEGF-All, a precursor to XC001 in which VEGF121, VEGF165 and VEGF189 are expressed in an approximate 2:2:1 ratio, was shown to be more effective at inducing angiogenesis and hind limb blood flow than comparable vectors with cDNA for individual VEGF isoforms in the ischemic mouse hind-limb model. Administration of AdVEGF-All provided superior restoration of blood flow than did administration of the Ad vectors carrying cDNA coding for the individual isoforms across a specific dose range. The AdVEGF-All minimum effective dose (MED) based on muscle volume was approximately 10⁴ to 10⁵ vp (human equivalent dose [HED]˜10⁸ to 10⁹ pu). This study demonstrated that a mixture of multiple Ad viral vectors each with a transgene expressing a single VEGF isoform or an Ad viral vector with a transgene coding for multiple VEGF isoforms provided a significant improvement in hindlimb flow ratio (angiogenic response) compared to administration of an Ad vector with cDNA for a single isoform. This supports the conclusion that these individual isoforms function synergistically, and that use of such a multiple-isoform drug may yield even better clinical efficacy than did the AdVEGF121 precursor.

The improvement in angiogenic potency that was observed for AdVEGF-All led to the investigation of the impact of administering an altered ratio of the major VEGF isoforms that could potentially provide further optimization of the safety profile of a candidate drug for clinical development. The drug candidate, XC001, was constructed to increase the ratio of isoforms containing exon 6a. XC001 was found to provide a potent angiogenic response in a similar fashion to VEGF-All but was found to have a better safety profile as measured by mouse mortality after IV dosing (no deaths were noted at doses of XC001 that were approximately 10-fold greater than the highest proposed human dose), by slower tumor growth in a mass of Lewis lung carcinoma cells injected into mouse subcutaneous tissue after an IV injection of product, and by less pulmonary edema noted by lung weight after an intratracheal administration of AdVEGF vectors.

The translatability of the latter two artificial animal experiments to humans is unclear. In the mouse mortality study, XC001 and AdVEGF-All were administered intravenously at doses of 5×10⁹ and 5×10¹⁰ pu. The HED based on body surface area were approximately 2×10¹² and 2×10¹³ pu, respectively. All animals at the HED of 2×10¹³ died in both groups while no mortality for XC001 was observed at the HED of 2×10¹² and 66% died in the AdVEGF-All group. This yields a no-observed-effect-level (NOEL) for XC001 approximately 20-fold greater than the highest planned human dose. While causes of the deaths are unknown, it appears that VEGF levels may not fully explain it since liver VEGF levels were comparable between both groups. The mortality, especially at the highest dose, may in part be due to the high amount of adenovirus that accumulates in tissues such as the spleen and liver after intravenous administration in mice. These two organs contain many immune cells, including liver Kupffer cells, splenic dendritic cells and macrophages. These cells have been assumed to be responsible for the production of inflammatory cytokines/chemokines that cause activation of an innate immune response which could lead to death. It should be noted that intramyocardial XC001 delivery would be expected to result in less systemic product exposure than intravenous administration.

### XC001 Original IND Enabling Toxicology Study

To support the IND, a toxicology study was performed by the administration of XC001 to the hearts of adult Fisher 344 rats. The study was comprised of 21 groups of 10 animals/group (5 males and 5 females). Fifteen groups (150 rats) received acute coronary artery ligations immediately followed by injection of PBS (pH 7.4), AdNull vector (10⁷ pu), or XC001 (10⁵ vp (human equivalent dose (HED) of 4×10⁷), 10⁶ vp (HED of 4×10⁸) or 10⁷ vp (HED of 4×109)) divided into 5 uniformly distributed 20 μL intramyocardial injections in the wall of the left ventricle with sacrifice time points scheduled at 5, 14, and 30 days post-surgery and dosing. Six groups (60 rats) received no ligation but were administered either XC001 vector at 10⁵, 10⁶ or 10⁷ vp or PBS (pH 7.4) with sacrifice time points scheduled at 30 days and 1-year post-surgery and dosing.

There were no XC001 treatment-related deaths (of the 6 deaths, 4 occurred in animals that had received the coronary artery ligation and 2 occurred in non-ligated animals but none were associated with XC001), clinical observations, or effects on body weights, hematology, or serum chemistry over the course of the study. Injection of XC001 did not result in any pathological changes in the heart or any other organ system attributable to the vector at any dose level tested. The process of injection into the heart produced a range of changes from focal adhesions between the left lung and the pericardium, focal adhesions between the left thoracic wall and the left lung, and thickened pericardium, all expected in the context of the surgical intervention.

Examination of the brain, eyes, skin, fat, thymus, lung, pericardium, heart, liver, skeletal muscle-quadriceps, bone-femur, sciatic nerve, male and female reproductive organs, urinary bladder, spleen, pancreas, kidney, stomach and intestinal tract, and lymph nodes showed no treatment-related changes. The lack of any positive troponin results indicated that no serious persistent damage to the myocardium was induced due to the vector. Overall, intramyocardial administration of XC001 at doses up to 10⁷ vp to adult Fisher 344 rats with or without induced myocardial infarction was well tolerated with no adverse effects of treatment at 5, 14, or 30 days post-surgery and dosing.

In addition, intramyocardial administration (without coronary artery ligation) of XC001 followed by a 1-year observation period did not result in any changes to treatment on gross pathology, histopathology, hematology and serum chemistry. Other lesions observed were consistent with naturally occurring pathological processes commonly observed in rats and were not considered to be associated with treatment or other experimental manipulations.

### Supplemental Toxicology Study

A bridging toxicology study in normal rats was conducted to evaluate the toxicity of XC001 following single administration into the myocardium of Fischer Rats over 91 days. Animals were administered a total of 5 myocardial injections into the free wall of the left ventricle to yield the following doses: Group 1 was vehicle only (formulation buffer), Group 2 was 1×10⁷ vp (HED of 4×10⁹), Group 3 was 2.5×10⁸ vp (HED of 1×10¹¹), this represents the high dose in the clinical trial, and Group 4 was 2.5×10⁹ vp (HED of 1×10¹²).

The in-life parameters (daily general health observations, clinical observations, and body weights) assessed throughout the study duration were used to help support the study objective from a clinical perspective. The results of these parameters revealed no significant differences between groups, and no findings were of clinical concern. Coagulation, clinical chemistry, and hematology results yielded no significant differences between groups and sexes. Human VEGF was not detected in rat plasma in the Day 8, Day 30, or Day 90 cohorts. Gross necropsy findings did not reveal any abnormalities attributed to a specific testing group. Organ weights and organ weight to terminal body weight ratios also revealed no significant differences between groups and sexes.

In the histopathology analysis of the Day 8 animals, the only relevant positive microscopic observation consisted of the finding in some animals of chronic inflammation of the myocardium, defined as the infiltration of mixed mononuclear cells (lymphocytes and macrophages) and variable amounts of fibroplasia/fibrosis, mostly involving the free wall of the left ventricle where the injections were given. The inflammation occurred in a dose response manner with Group 3 and Group 4 animals possessing an increased incidence and severity of inflammation as compared to Group 2 and Group 1. In Group 3, which represents the HED of the highest planned dose in the clinical trial, only minimal to mild inflammation was observed while in Group 4, which has a HED 10-fold higher than the highest planned clinical dose, inflammation varied from mild to marked severity. However, the incidence and severity was reduced by Day 30, with only minimal/mild inflammation found in Group 1-3 animals, and a greater degree of inflammation (moderate) found in only one of ten Group 4 animals. The inflammatory process involved the free wall of the left ventricle, i.e. the site of experimental injection, and did not affect myocardium of interventricular septum, right ventricular free wall, or right or left atria. In moderate to marked instances (seen only in Group 4), chronic inflammation affected much of the left ventricular free wall (site of injections) and was present transmurally. An additional observation on Day 29/30 was a trend for increasing amounts of fibrous tissue within regions of the myocardium affected by the chronic inflammatory lesion. For the Day 90 cohorts, XC001-related changes consisted of fibrosis of the myocardium in male and female rats in Groups 3 and 4. Mononuclear cells in the myocardium was also a common finding in hearts from rats on Day 90 and occurred in all treatment groups (including controls; Group 1). This is a well-recognized, age-related spontaneous finding in rats and was not caused by treatment with XC001. Observations on Days 30 and Day 90 cohort animals were consistent with resolution of XC001-induced inflammatory lesions found in the myocardium on Day 8. No other tissues analyzed in Day 8, Day 30, or Day 90 cohort animals revealed abnormalities specific to a testing group.

Serum Cardiac Troponin I (cTnI) results did not demonstrate a correlation between dose escalation and increased cTnI values in Day 8, Day 30 and Day 90 cohorts. Since cTnI is a biomarker to indicate cardiac muscle injury, elevated serum cTnI values would be expected in those animals with an increased severity of chronic inflammation of the myocardium. However, this was not the case and in some instances the animals with the highest levels of cTnI had no to minimal inflammation of the myocardium.

In conclusion, intra-myocardial administration of XC001 was associated on Day 8 with chronic inflammation of the myocardium involving the free wall of the left ventricle (i.e. the site of experimental injection) that increased in incidence and severity with increasing dose of XC001. Observations on Days 30 and 90 were consistent with resolution of XC001-induced inflammatory lesions found in the myocardium on Day 8. In addition, all in-life parameters, clinical pathology, serum cTnI, plasma VEGF, and necropsy observations revealed no signs of clinical concern correlating to any of the testing groups.

### Clinical Data

A wide range of clinical experience has been obtained for adenovirus and VEGF isoforms. Adenovirus vectors have properties that make them ideal for the delivery of VEGF genes for therapeutic angiogenesis, namely, effective transduction of cardiovascular tissues, nonintegration into the human genome and short-term transduction. Most importantly, these vectors have an extensive track record for human gene therapy and a demonstrated safety profile at the doses being evaluated. Moreover, long-term safety (out to a median of 11.8 years post gene therapy) of VEGF isoforms with adenovirus delivered into the heart has been demonstrated. However, several attempts to use the gene encoding for VEGF in the clinic have met with limited success for a variety of potential reasons including ineffective delivery route, ineffective gene vectors, and poor choice of efficacy endpoint criteria:
Delivery by intracoronary infusion which is several logs less effective in animals in delivering therapeutic agents to the heart compared to intramyocardial delivery.
Delivery via indirect, endocardial injection using the NOGA guidance catheter system is predicted to deliver to only 50-60% of targeted area and is considered highly inaccurate by investigators. Importantly, with the intraventricular route, there is the risk that the vector will be injected intravascularly, with the attendant risk of anti-vector innate immunity and a serious adverse event.
Use of plasmid DNA rather than viral vector affords much less efficiency than virus with likely differences in duration of expression.
Choice of single-photon emission computed tomography (SPECT) as primary endpoint in some trials is a likely limitation. SPECT myocardial perfusion imaging has multiple limitations, including relatively long acquisition protocols and considerably poorer spatial resolution than other available modalities, limiting detection of sub-endocardial perfusion defects. Furthermore, the discordance of tracer uptake (tracer uptake does not correlate with myocardial blood flow) at higher myocardial blood flows limits sensitivity in detecting mild to moderate stenosis.

However, both proof of concept in preclinical investigations as well as positive confirmation of effect in the clinic have been demonstrated for a VEGF gene delivery strategy using prototype gene therapy candidate AdVEGF121 wherein an Ad5 vector coding for a single isoform, VEGF121, was delivered directly into the myocardium via mini-thoracotomy. The mini-thoracotomy/epicardial route of administration provides absolute control of the sites of myocardial injection, limits inadvertent intravascular administration, and has been shown to be safe in a small dose escalation Phase 1 trial and in the Phase 2 REVASC trial.

In terms of efficacy, time to 1 mm ST-segment depression as well as total exercise duration and time to moderate angina and in angina symptoms as measured by the CCS Angina Class and SAQ were all improved by VEGF gene transfer.

In these two trials there was no evidence of systemic or cardiac-related adverse events related to the vector. In the Phase 1 trial, there were 3 deaths reported among a group of patients that received AdVEGF121 while undergoing CABG via a median sternotomy while no deaths were reported in patients receiving only intramyocardial AdVEGF121 via a thoracotomy similar to the procedure planned in the current study. The authors reported that there was no evidence of systemic or cardiac-related adverse events related to vector administration. Two of the deaths were most likely related to CABG surgery and their advanced CAD while the third patient experienced a sudden death of unknown cause. In the REVASC trial during 12 months of follow-up, there were two deaths out of 32 subjects treated with AdVEGF121 compared to one among the 35 subjects in the placebo group. While causality was not mentioned in the publication, the first author stated that both subjects treated with AdVEGF121 had severe ischemic disease which, when coupled with post-procedure complications, likely contributed to the poor outcomes that were not attributed to AdVEGF121 itself. The publication also reported 4 serious cardiac events that were considered related to the procedure. However, only 3 patients in the AdVEGF121 group, as compared to 9 patients in the control group, experienced major cardiac events after the initial 3 weeks, possibly consistent with late benefits of therapeutic angiogenesis. In a meta-analysis of randomized controlled trials (RCTs) that compared VEGF gene therapies (including REVASC) and standard treatments in CAD (mean 6 months of follow-up) a decreased risk of serious cardiac events (MI, acute coronary syndrome, cardiac arrest, cardiogenic shock, heart failure, and surgical cardiac interventions) was demonstrated and the use of adenoviral vectors to deliver VEGF showed more potential benefit in terms of the risk of serious cardiac events while no difference was noted on mortality. In addition, an 8-year follow-up of intracoronary Ad-VEGF-A165 revealed an incidence of major adverse cardiovascular events that did not differ from a placebo control group.

While Ad vectors are considered immunogenic, dose and route of administration are key factors to an immune response. Intravenous administration as opposed to an intramuscular injection would be expected to result in more systemic exposure and potentially a higher probability of an immune reaction. A large body of nonclinical and clinical data for Ad vectors yielding VEGF121 and VEGF165 transgene products with doses up to 4×10¹⁰ vp has not elicited a clinically meaningful immune related safety trend or issue with follow-up to a median of 11.8 years. In mice, it has been shown that systemically administered Ad vectors are rapidly cleared from the blood of mice, with a half-life of less than 2 minutes, with large accumulation in the liver and spleen. These two organs contain many immune cells, including liver Kupffer cells, splenic dendritic cells and macrophages and these cells have been assumed to be responsible for the production of inflammatory cytokines/chemokines responsible for activation of an innate immune response. In the above-mentioned dose escalation Phase 1 study of an intramyocardial AdVEGF121 injection (dose up to 4×10¹⁰ pu), shedding of vector or wild-type Ad was not detected in any sample (Days 2, 4, and 7) from any site (nose, throat, urine, and blood) in any subject. Furthermore, plasma VEGF levels were not above baseline values beyond Day 3 post administration. However, serum anti-Ad5 neutralizing antibody levels were increased in all individuals, although more so in patients with higher pretherapy anti-Ad5 neutralizing antibodies. In the REVASC trial of an intramyocardial AdVEGF121 injection (4×10¹⁰ pu), urine and throat swabs for adenoviral cultures evaluated at approximately Day 7 for 23 of the VEGF treated patients were all negative. Therefore, based on a large body of clinical and nonclinical data, there appears to be a low risk for a clinically relevant immune reaction at the doses being studied in the current trial.

### Rationale for Trial

CAD is a chronic disease in which blood flow is obstructed through the coronary arteries that supply the heart with oxygen-rich blood leading to ischemia. Untreated, CAD usually continues to worsen. Many CAD patients have symptoms such as chest pain (angina) and fatigue, which occur when the heart is not receiving adequate oxygen. As many as 50% of patients, however, experience no symptoms until a heart attack occurs. CAD remains the leading killer of men and women in the world. Ischemic conditions of the heart require therapeutic intervention, including pharmacologic, coronary artery stenting and cardiac surgical bypass. However, there is a significant population with CAD who have refractory angina secondary to obstructive CAD, in which these interventions no longer will be effective or cannot be used. Preclinical studies of exogenous delivery of the VEGF121 and 165 isoforms using an adenovirus vector have demonstrated the capacity of this therapy to re-vascularize cardiac and skeletal muscle and alleviate ischemia. Safety of intramyocardial delivery of adenovirus with the transgene expressing the isoform VEGF121 has been established in several human trials and preliminary efficacy of AdVEGF121 looks promising. In marked contrast to Ad vectors expressing individual isoforms of VEGF, in preclinical studies with an ischemic hind-limb model XC001, an Ad5 vector expressing the cDNA/genomic hybrid of the VEGF gene, mediated nearly full recovery of blood flow at a dose of two logs less than required for the previous clinical vector AdVEGF121. Thus, XC001 is not only closer to the natural expression of VEGF in the heart, but it is more powerful (per vector) than that used in prior clinical studies and is therefore likely to have an improved safety profile. The proposed Phase ½ clinical trial will be used to determine the safety and tolerability of direct administration of the vector XC001 to the ischemic myocardium and to generate evidence regarding whether direct administration of XC001 to the ischemic myocardium will induce growth of collateral blood vessels and improve cardiac function and QOL.

### Rationale for Proposed Doses

XC001 will be administered as a one-time therapy by TECAP to allow direct delivery of the vector to the target tissue compartment. This replicates the route of administration used in the nonclinical and clinical studies and data suggests this procedure is much more effective at delivering vector than intracoronary or endocardial catheter administration. Prior to the procedure, each subject will have their medical history, physical exam and other assessments reviewed by a team of cardiologists and cardiovascular surgeons for consensus on the suitability of the candidate for the trial.

A wide range of clinical experience has been obtained for Ad vectors and VEGF isoforms 121 and 165, and these vectors have an extensive track record for human gene therapy given intramyocardially with a demonstrated safety profile. Moreover, long-term safety (out to a median of 11.8 years after gene therapy) of VEGF isoforms with adenovirus delivered into the heart has been demonstrated (Table 5). Other isoforms containing exon 6a do not appear to have been studied in humans but as discussed above all exons except for exon 6 are represented in VEGF165. In light of the VEGF121 and VEGF165 human experience and since exon 6a containing isoforms are naturally occurring and expected to have fewer systemic effects than VEGF121 due to binding to the extracellular matrix, it is believed to be unlikely that XC001 poses a safety risk beyond that of Ad vectors expressing VEGF121 or VEGF165.

The proposed starting dose in humans, 1×10⁹ vp, is considered safe given the totality of nonclinical safety pharmacology and toxicology data with XC001. The safety of the second to fourth XC001 doses (1×10¹⁰, 4×10¹⁰ and 1×10¹¹ vp) is supported by the totality of the nonclinical and clinical experience of Ad vectors containing VEGF isoforms and by the XC001 toxicology studies. Subjects will be monitored in the hospital for the first one to two days post XC001 administration (or longer if deemed necessary). The XyloCor medical monitor will closely monitor all AEs/SAEs as they emerge. Within cohorts, an internal safety group will review all available safety data, including the Day 7 visit of the latest subject dosed in the cohort, before any decision is made to dose another subject. If no adverse trends are observed, dosing of the next subject will commence. The AE/SAE profile may also require the external Independent Data Monitoring Committee (IDMC) to be part of the decision to dose the subsequent subject. In addition, between cohorts, the IDMC will be reviewing all available safety data, including that of the third subject in the last cohort, up to and including their Day 7 visit, before any subsequent subject is dosed. The one-week dosing interval is considered appropriate given that potential safety findings from the procedure would have been expected to occur perioperatively and the kinetics of expression would have given peak systemic levels of the transgene product. For the latter, clinical data shows maximal VEGF expression between 48-72 hours post intramyocardial dosing and a lack of Ad vector shedding is observed 2 days post administration. XC001 would be expected to be efficacious at 1×10⁹ vp since the estimated MED from the mouse hindlimb model has a HED between approximately 10⁸ to 10⁹ vp (Table 3). Efficacy at this dose is also supported by the positive efficacy observed in the REVASC trial (AdGVVEGF121.10 at 4×10¹⁰ pu) and by findings in nonclinical studies that suggest XC001 may be logs more potent than AdVEGF121.

After the third subject in the fourth cohort is dosed and attends their Day 7 visit, all cumulative safety data will be reviewed by the IDMC in order to make a recommendation to XyloCor on whether and when to proceed with dosing of approximately 17-21 additional subjects at the highest tolerated dose. The rationale behind adding additional subjects at the highest tolerated dose after dose escalation relates to the enhanced ability to detect some degree of efficacy as well as additional safety that will assist in selecting a dose for further study with greater confidence in the risk-benefit analysis. With 3 subjects per cohort, important safety and tolerability information is anticipated but only a trend in some efficacy parameters would be expected. Adding approximately 17-21 subjects to the highest tolerated dose (N=20-24 at this dose) will enable an examination of a set of loosely correlated outcome measures of ischemia (i.e., time to ST segment depression on exercise tolerance test; total perfusion deficit, myocardial blood flow and coronary flow reserve by PET; angina episodes; ischemic burden by ambulatory ECG, etc.) to make an assessment of preliminary evidence of efficacy. These data should provide a richer dataset to check certain assumptions on the treatment effect and allow for more confidence in the dose taken forward for further development.

### Overall Study Design

This is a 6-month (with 6-month extension) Phase ½, first-in-human, multicenter, open-label, single arm dose escalation trial of XC001. No control group is included. Approximately 12 subjects (N=3 per cohort) who have refractory angina will be enrolled into 4 ascending dose groups (1×10⁹, 1×10¹⁰, 4×10¹⁰ and 1×10¹¹ vp of XC001), followed by an expansion of the highest tolerated dose with approximately 17-21 additional subjects. XC001 will be administered via TECAP directly to the free wall of the left ventricle of subjects.

After qualifying for the study based on entry criteria and assessed by both the study cardiologist and surgeon, the Eligibility Review Committee (ERC) will review each candidate's past medical history and screening assessments and formally clear each candidate for inclusion into the trial. Subjects will be monitored in the hospital for the first one to two days post XC001 administration (or longer if deemed necessary). The medical monitor will closely monitor all AEs/SAEs as they emerge. Within cohorts, an internal safety group will review all available safety data, including the Day 7 visit of the latest subject dosed in the cohort, before any decision is made to dose another subject. If no adverse trends are observed, dosing of the next subject will commence. The AE/SAE profile may also require the external Independent Data Monitoring Committee (IDMC) to be part of the decision to dose the subsequent subject. In addition, between cohorts, the IDMC will be reviewing all available safety data, including that of the third subject in the last cohort, up to and including their Day 7 visit, before any subsequent subject is dosed. At any given IDMC meeting, the IDMC may recommend stopping the trial, dosing additional subjects at the current dose, proceeding to the next dose cohort, or proceeding by dosing additional subjects at a lower dose (further details are provided in the IDMC charter). After the third subject in the fourth cohort is dosed and attends his/her Day 7 visit, all cumulative safety data will be reviewed by the IDMC in order to make a recommendation to XyloCor on whether and when to proceed with dosing of additional subjects at the highest tolerated dose.

### Description of Investigational Product (IP)

The investigational product XC001 is composed of the active ingredient AdVEGFXC1, a replication-deficient adenovirus serotype 5 vector containing a cDNA/genomic hybrid cassette coding for multiple isoforms of the vascular endothelial growth factor proteins. Up to 4 doses will be studied: 1×10⁹, 1×10¹⁰, 4×10¹⁰ and 1×10¹¹ vp of XC001. The route of administration will be one-time intramyocardial injections directly into the free wall of the left ventricle by TECAP. Total volume of investigational product administered will be 1.5 mL.

IP will be delivered to the operating room as two sterile bags packaged in a non-sterile outer bag which will then be placed in a container for transport from Investigational Drug Service (IDS) to the operating room (OR). One sterile bag will contain the 14 syringes that are prefilled with 0.1 mL of prepared XC001, with the other sterile bag containing the 3 syringes prefilled with 0.2 mL of prepared XC001. Each sterile bag will be labelled according to institutional practice. The three prefilled syringes are slightly overfilled with 0.2 mL of IP to allow removal of any air bubbles and proper priming of the needle just prior to injection. Three 27-gauge spinal needles will also be provided. The injection volume will be 0.1 mL per each of the 15 intramyocardial injections distributed across the free wall of the left ventricle as described in further detail below.

This protocol has an ascending dose escalation study design where a subject is assigned to 1 of 4 possible dose cohorts expressed as viral particles (vp) of AdVEGFXC1: 1×10⁹ vp, 1×10¹⁰ vp, 4×10¹⁰ vp and 1×10¹¹ vp. In the expansion phase of the trial, all subjects will receive the highest tolerated dose as determined from the escalation phase. The dose assignment and dilution worksheets will have been provided to the investigational drug pharmacist who will have the primary responsibility for receipt, short-term storage, thawing, dilution and prefilling the syringes according to Biosafety Level 2 (BSL-2) practices and usual institutional practice for parenteral sterile compounding that will include maintaining external sterility of each syringe and needle or syringe cap so that they may enter the sterile field in the operating room.

It is important that the preparation in the investigational drug service is proactively coordinated with the activities in the operating room within the stability parameters labeled for the investigational product, specifically, dosing should occur within 7 hours of removal of the drug product from the freezer. The site coordinator will alert the site Pharmacist as to when to start preparing IP (note that it may take up to 1.5 hours to prepare IP).

The final investigational product will be provided in 3 mL borosilicate glass vials, with a fill volume of approximately 1.2 mL (extractable volume not less than 1.0 mL), sealed with latex-free stoppers and aluminum caps. Each cryovial will be labeled with the product name, concentration, fill volume and vial number; route of administration; statement “Caution: New Drug—Limited by Federal law to Investigational Use;” storage conditions; lot number; and manufacturer.

### Administration

The subject is placed in a 30° decubitus position supine with a roll under the back and the arm out 90 degrees on an arm rest to provide access to the pleural space from a more anterior approach and defibrillator patches are placed on the chest. The surgeon stands facing the subject's heart with the camera-holding assistant on the same side when filming is utilized. The television monitor should be positioned so that the surgeon, the left ventricle of the subject and the monitor are aligned to allow the surgeon to look straight ahead when operating.

Follow a minimally invasive surgical approach for transthoracic epicardial access. A 4-5 cm anterolateral incision is made in the 5th to 7th intercostal space (based upon relevant imaging studies to provide best access to the heart just basal to the cardiac apex). This incision will typically be in the inframammary crease in women. A Tuffier Retractor (or any self-retaining retractor) is inserted. Adhesions from the lung and chest wall are taken down with electrocautery. A port may be inserted into the 7th or 8th intercostal space at the anterior axillary line for the thoracoscope if utilized or the thoracoscope can be inserted directly through the primary incision. The pericardium is then opened longitudinally 1 cm anterior to the phrenic nerve. If there has been a previous sternotomy, the pericardium may need to be dissected off of the epicardial surface to create a pericardial plane. This is typically easily performed, but injections may be performed trans-pericardially if dissection is deemed a prohibitive risk (needle depth and right angle placement should be adjusted accordingly). Do not attempt trans-pericardial injections unless it is determined that opening/taking down the pericardium poses an undue risk. In this case, trans-pericardial injections can be undertaken with great care, adjusting the depth of the needle distal to the right-angle clamp to account for the pericardial thickness. The coronary arteries and veins should be avoided during injection. This can be facilitated by aspiration of the syringe prior to injection, which will also confirm that injections are not occurring in the ventricular chamber. The injections are then performed according to the procedure that follows. Once the procedure is completed direct intercostal nerve blocks could be considered with Exparel or Marcaine, and if thoracoscopy ports are placed complete cryoablation of intercostal nerves for pain control could be considered.

Start the video recorder (if applicable) just prior to beginning the microinjections. An injection grid will be planned prior to the surgery.

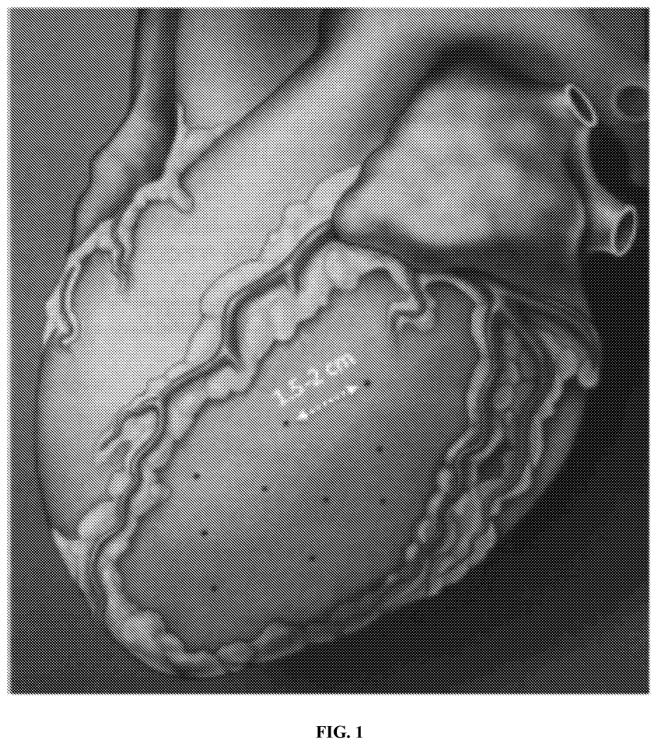

As a first principle, the left ventricle should be blanketed with a total of 15 microinjections of 0.1 mL each of investigational product separated approximately 1.5 to 2 cm from each other (FIG. 1). The surgeon should emphasize injections in areas that are known to be ischemic based on all clinical information, where collateral vessel formation could potentially provide the greatest benefit. Clear cut areas of scar and thinning should be avoided.

A long right-angled hemostat forcep is placed approximately 4-7 mm from the tip of the spinal needle to control depth of injection and to stabilize the needle over >5 beats to allow maximum absorption of investigational product. Obliquely aimed injections may help prevent less outflow when needle is removed. For the first injection, one of the prefilled syringes slightly overfilled with 0.2 mL will be used so that a 27-gauge spinal needle can be attached and primed to allow removal of any air bubbles and eliminate any dead space (only 0.1 ml is to be injected). For the remaining 14 injections, the prefilled syringes (0.1 ml) will utilize the same 27-gauge spinal needle (with right-angled forcep attached). Blank injection maps will be provided to help with pre-surgery injection planning and for noting any issue with the injections in the OR.

After all injections are performed, the heart should be inspected for any sites of needle hole bleeding. Digital pressure should be applied to any injection site with persistent bleeding. Once hemostasis is achieved, the thoracoscope is removed. A chest tube is then inserted through the port site or a separate incision and connected to a Pleurovac. The thoracotomy incision is closed in layers with absorbable suture and a sterile dressing placed over the incision. Anesthesia will be discontinued, and subject cared for following institutional guidelines for post-anesthesia care.

### Assessment of Efficacy

### Modified Bruce Protocol Exercise Tolerance Test

For the primary efficacy outcome measure of time to onset of 1 mm ST segment depression, a treadmill exercise protocol, modified from the standard Bruce method to start at a lower workload than the standard test, will consist of multiple stages of progressively greater workloads created by increasing the percent grade and speed of the treadmill while monitoring cardiac function. As part of the baseline measurement, testing will be performed twice during the screening period with each test separated in time by at least 72 hours or longer. In order to be eligible for the trial, the subject must be able to exercise for 90 seconds to approximately 9 minutes while exhibiting ≥1 mm horizontal or down-slopping ST segment depression on at least one of the tests, with the other test demonstrating >0.5 mm ST segment depression. The ST segment requirement will apply to subjects in cohort 4, as well as the subjects in the expansion phase. The ST segment requirement will not apply for subjects in cohorts 1, 2 and 3. Subjects will be instructed to withhold taking anti-anginal medication the morning of their assessment if such medication is normally taken in the morning. Any short-acting NTG should be withheld within 4 hours of the assessment. If short-acting NTG is taken during this period or the patient is not in their usual state of health, the subject will be instructed to inform the site staff and reschedule the ETT.

A detailed ETT protocol and independent review charter will be provided by a third-party, blinded ETT core laboratory with all the specifications on general requirements, staffing, equipment including maintenance and calibration, and test termination. The ETT core laboratory will be responsible for training and certification of the nurse or technician that will performing the test on subjects. Ideally, a primary nurse or technician and one back-up is identified for the duration of the trial. The ETT core laboratory interpretation (blinded assessor) and analysis of each test will be used for all efficacy analyses of the trial. In addition, the ETT core laboratory must review and approve the baseline paired ETT and confirm eligibility.

### Myocardial Perfusion Imaging by Positron Emission Tomography

Regional and global myocardial perfusion will be assessed using PET imaging in accordance with the study-specific acquisition protocol. PET scans will be performed using a whole-body PET scanner. Anti-hypertensives and beta-blockers, and calcium channel blockers will be withheld on the morning of the scan. Subjects will be allowed to continue using sublingual nitroglycerin as needed. Studies will be performed after 4 hours of fasting and 24 hours of abstinence from caffeine-containing products. The PET scan will take approximately 2.5 hours, including subject preparation.

Myocardial perfusion will be assessed at rest and during maximal hyperemia using a standard adenosine or regadenoson infusion, and 13N-ammonia or 82Rubidium as the flow tracer. After transmission imaging and beginning with the intravenous (IV) bolus administration of 13N-ammonia [(˜10-20 millicurie (mCi) or 82-Rubidium (˜10-60 mCi)], list mode images will be acquired for approximately 19 minutes (13N-ammonia) or 7 minutes (82Rubidium). Fifteen or thirty minutes later, subjects will undergo a standard infusion of adenosine (0.14 mg/kg/min for 4 minutes) or regadenoson (0.4 mg bolus injection). At peak hyperemia, a second dose of 13N-ammonia (˜10-20 mCi) or 82Rubidium (˜10-60 mCi) will be injected IV, and images recorded in the same manner. The heart rate, blood pressure, and 12-lead ECG will be recorded at baseline and throughout the infusion of adenosine or regadenoson, and at recovery.

All PET scans will be done for research (non-clinical) purposes only. For safety reasons, all PET scans will be reviewed locally by the site investigator, or his/her designee, for clinically important findings. The Screening PET will be read locally as part of the ERC packet. No reports or analyses will be provided to sites from the PET core laboratory and studies will not be assessed in real-time. The PET core laboratory will provide the following services: qualification of site equipment and study technologists; development of an imaging acquisition protocol and quick reference guide for study personnel; development of an independent review charter describing the processes, services and image interpretation; site technical training, certification and ongoing support during the conduct of the trial; tracking of imaging studies and quality review; quantitative analysis and independent overreading of all imaging studies as described below; and data management and data transfer services of the final data.

A complete quantitative analysis of rest and stress myocardial perfusion PET images will include the following:

### Semi-Quantitative Myocardial Perfusion Analysis

Total Perfusion Deficit (TPD) measures the total left ventricular perfusion deficit at rest (reflecting scarred myocardium) and during stress (reflecting both scarred+ischemic myocardium), as well as the difference between stress and rest (reflecting ischemic myocardium). TPD scores will be processed using standard software.

For each subject, the following variables will be obtained at baseline and during the follow-up scans: (a) rest TPD—individual values will be obtained for each of the coronary vascular territories (left anterior descending, LAD; left circumflex, LCx; and right coronary artery, RCA) and also for the entire left ventricle (LV) (global rest TPD); (b) peak hyperemic-stress TPD—individual values will be obtained for each of the coronary vascular territories (LAD, LCx and RCA) and also for the entire LV (global stress TPD); and (c) difference TPD—individual values will be obtained for each of the coronary vascular territories (LAD, LCx and RCA) and also for the entire LV (global difference TPD).

### Quantification of Left Ventricular Function

Rest and post-stress left ventricular ejection fraction (LVEF) will be calculated from the gated myocardial perfusion images using commercially available software. For each subject, the following variables will be obtained at baseline and during the follow-up scans: rest LVEF and post-stress LVEF.

### Ambulatory Electrocardiography

Transient ST-segment deviation will be monitored by continuous ambulatory ECG for a period of 5 days as indicated on the Schedule of Assessments. The 5-day ambulatory ECG should be performed within the specified window of the nominal visit. The Day 1, or baseline, ambulatory ECG must be performed during any 5-day period in the screening period just prior to Day 1. Because most ischemic episodes during routine daily activities are related to increases in heart rate, it will be essential to encourage similar daily activities at the time of each ambulatory ECG recording. An ambulatory ECG monitoring core laboratory will provide an ambulatory ECG recorder in the form of an ePatch device. The core lab will also provide site technical training on its use and placement, a procedure manual and quick reference guide for study personnel, ongoing support during the trial, tracking of ambulatory ECG studies and quality review, blinded analysis in a written independent charter, data management and data transfer services of the final data. The criteria for an ischemic episode will be ≥1 mm of horizontal or down-sloping ST segment depression lasting ≥1 minute and separated from another episode by ≥1 minute. The maximal depth of the ST segment depression during each episode will be noted to allow the calculation of an index of ST segment depression (mm) times duration (min) as the “total ischemic burden.”

### Actigraphy

A motion biosensor device (activity tracker) will be provided to subjects to wear for 14 days to measure gross motor activity. The 14-day period should occur in advance of the study visit indicated in the Schedule of Assessments so that it concludes by the time of the visit and the device can be returned for interpretation by the actigraphy core laboratory. An actigraphy core laboratory will provide study personnel training and 24/7 technical service and support, study guide, device rental, motion assay services and data analysis in a written independent review charter.

### Quality of Life (QOL)

The Seattle Angina Questionaire (SAQ) is the most sensitive, specific and responsive health-related quality of life instruction for coronary artery disease. The SAQ is self-administered and has been shown to be valid, reproducible and sensitive to clinical change. The SAQ quantifies subjects' physical limitations caused by angina, the frequency of and recent changes in their symptoms, their satisfaction with treatment, and the degree to which they perceive their disease to affect their quality of life. Each scale is transformed to a score of 0 to 100, where higher scores indicate better function (eg, less physical limitation, less angina, and better quality of life). The instrument has 19 items that yields five subscale scores: physical limitation, angina stability, angina frequency, treatment satisfaction and disease perception. A change in 10 points in any of the subscales is considered to be clinically important.

The EQ-5D-3L QOL instrument essentially consists of 2 pages: the EQ-5D descriptive system and the EQ visual analogue scale (EQ VAS). The EQ-5D-3L descriptive system comprises the following five dimensions: mobility, self-care, usual activities, pain/discomfort and anxiety/depression. The EQ VAS records the patient's self-rated health on a vertical visual analogue scale.

The Clinical Global Impression (CGI) is broken into two parts. The Clinical Global Impression—Severity score, collected at baseline, consists of a single question completed by the investigator: “Relative to the past 7 days how is the patient's refractory angina: 1=Normal—not at all ill, symptoms of disorder not present in the past seven days; 2=Borderline ill—subtle or suspected pathology; 3=Mildly ill—clearly established symptoms with minimal, if any, distress or difficult in social and occupational function; 4=Moderately ill—overt symptoms causing noticeable, but modest, functional impairment or distress; symptom level may warrant medication; 5=Markedly ill—intrusive symptoms that distinctly impair social/occupational function or cause intrusive levels of distress; 6=Severely ill—disruptive pathology, behavior and function and frequently influenced by symptoms, may require assistance from others; 7=Among the most extremely ill patients—pathology drastically interferes in many life functions.

The Clinical Global Impression—Improvement score consists of a single question completed by the investigator: “Compared to the patient's condition at baseline, this patient's refractory angina is: 1=very much improved since the initiation of treatment; 2=much improved; 3=minimally improved; 4=no change from baseline (the initiation of treatment); 5=minimally worse; 6=much worse; 7=very much worse since the initiation of treatment.”

### Angina and Prophylactic Nitroglycerine (NTG) Use Log

Subjects will be given a paper diary to collect angina episodes and specifics about each episode (triggers, severity, treatments, etc.). There will also be a prophylactic nitroglycerine use diary. Subjects will record their anginal episodes as well as NTG use during the following intervals: 14-days prior to Day 1 visit to serve as the baseline with diary collected on Day 1; 14 days prior to the Month 3 visit with diary collected at the Month 3 visit; 14 days prior to the Month 6 visit with diary collected at the Month 6 visit; and the 2 week (14 days) period prior to the Month 12 visit. Diary collection should coincide with when the subject is wearing the activity tracker except for the Month 12 visit.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Enumerated Embodiments

The following enumerated embodiments are provided, the numbering of which is not to be construed as designating levels of importance.

Embodiment 1 provides a method of treating a cardiovascular disease in a subject in need thereof, the method comprising administering directly into the heart of the subject during Transthoracic Epicardial Procedure (TECAP) an effective amount of pharmaceutical composition comprising a viral vector comprising a therapeutic polynucleotide.
Embodiment 2 provides the method of embodiment 1, wherein the pharmaceutical composition is administered through a series of 15 injections at separate delivery sites in the heart of the subject, and wherein the viral vector diffuses through substantially all of the heart.
Embodiment 3 provides the method according to any one of Embodiment 1 or Embodiment 2, wherein the viral vector is an adenoviral vector.
Embodiment 4 provides the method according to any one of Embodiments 1-3, wherein the viral vector comprises a polynucleotide encoding one or more isoforms of VEGF.
Embodiment 5 provides the method according to any one of Embodiments 1-4, wherein the heart of the subject is visualized throughout the procedure using a thorascope.
Embodiment 6 provides the method according to claims 1-5, wherein a dose of the viral vector of about 1×10⁹ vp, about 1×10¹⁰ vp, about 4×10¹⁰ vp or about 1×10¹¹ vp is administered.
Embodiment 7 provides the method of Embodiment 2 wherein each injection has an injection volume of about 0.1 mL.
Embodiment 8 provides the method according to any one of Embodiments 1-8, wherein the cardiovascular disease is coronary artery disease.
Embodiment 9 provides the method according to any one of Embodiments 1-8, wherein the TECAP comprises making a 4-5 cm anterolateral incision in the 5th to 7th intercostal space of the subject.
Embodiment 10 provides the method according to any one of embodiments 1-9, wherein the injections are made in the left ventricle.

1. A method of treating a cardiovascular disease in a subject in need thereof, the method comprising administering directly into the heart of the subject during Transthoracic Epicardial Procedure (TECAP) an effective amount of pharmaceutical composition comprising a viral vector comprising a therapeutic polynucleotide.

2. The method of claim 1, wherein the pharmaceutical composition is administered through a series of 15 injections at separate delivery sites in the heart of the subject, and wherein the viral vector diffuses through substantially all of the heart.

3. The method according to any one of claim 1, wherein the viral vector is an adenoviral vector.

4. The method according to claim 1, wherein the viral vector comprises a polynucleotide encoding one or more isoforms of VEGF.

5. The method according to claim 1, wherein the heart of the subject is visualized throughout the procedure using a thorascope.

6. The method according to claim 1, wherein a dose of the viral vector of about 1×10⁹ vp, about 1×10¹⁰ vp, about 4×10¹⁰ vp or about 1×10¹¹ vp is administered.

7. The method of claim 2 wherein each injection has an injection volume of about 0.1 mL.

8. The method according to claim 1, wherein the cardiovascular disease is coronary artery disease.

9. The method according to claim 1, wherein the TECAP comprises making a 4-5 cm anterolateral incision in the 5th to 7th intercostal space of the subject.

10. The method according to claim 1, wherein the injections are made in the left ventricle.


SEQUENCE LISTING (1 sequence)

SEQ ID NO: 1 - DNA, 35371 bp, Artificial Sequence
  DNA sequence of AdVEGFAll6A+
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgggccccccctcgaggtcgacggtatcgataagcttgatatcgaattcctgcagtcaccgtcgtcgacggtatcgataagcttgatatcgaattccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccgatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagctcgacaagaaaagtaagtggccctgactttagcacttctccctctccatggccggttgtcttggtttggggctcttggctacctctgttgggggctcccatagcctccctgggtcagggacttggtcttgtgggggacttgtggtggcagcaacaatgggatggagccaactccaggatgatggctctagggctagtgagaaaacatagccaggagcctggcacttcctttggaagggacaatgccttctgggtctccagatcatttctgaccaggacttgctgtttcggtgtgtcagggggcactgtggacactggctcactggcttgctctaggacacccacagtggggagagggagtgggtggcagagaggccagcttttgtgtgtcagaggaaatggcctcttttggtggctgctgtgacggtgcagttggatgcgaggccggctggagggtggtttctcagtgcatgccctcctgtaggcggcaggcggcagacacacagccctcttggccagggagaaaaagttgaatgttggtcattttcagaggcttgtgagtgctccgtgttaaggggcaggtaggatggggtgggggacaaggtttggcggcagtaacccttcaagacagggtgggcggctggcatcagcaagagcttgcagggaaagagagactgagagagagcacctgtgccctgccctttcccccacaccatcttgtctgcctccagtgctgtgcggacattgaagcccccaccaggcctcaaccccttgcctcttccctcagctcccagcttccagagcgaggggatgcggaaaccttccttccaccctttggtgctttctcctaagggggacagacttgccctctctggtcccttctccccctcctttcttccctgtgacagacatcctgaggtgtgttctcttgggcttggcaggcatggagagctctggttctcttgaaggggacaggctacagcctgccccccttcctgtttccccaaatgactgctctgccatggggagagtagggggctcgcctgggctcggaagagtgtctggtgagatggtgtagcaggctttgacaggctggggagagaactccctgccaagtaccgcccaagcctctcctccccagacctccttaactcccaccccatcctgctgcctgcccagggctccaggacacccagccctgcctcccagtccaggtcgtgctgagcaggctggtgttgctcttggttccgtgccagctcccaaggtagccgcttcccccacaccgggattcccagaggttctgtcgcagttgcaaatgaaggcacaaggcctgatacacagccctccctcccactcctgctccccatccaggcaggtctctgaccttctccccaaagtctggcctaccttttatcacccccggaccttcagggtcagacttggacagggctgctgggcaaagagccttccctcaggctttgccccctgccggggactgggagccactgtgagtgtggagacctttgggtcctgtgccctccacccagtctcggcttcccaccaaagccttgtcaggggctgggtttgccatcccatggtgggcagcgtgaggagaagaaagagccatcgagtgcttgctgcccagacacgcctgtgtgcgcccgcgcatgcctccccagagaccacctgcctcctgacacttcctccgggaagcggccctgtgtggctttgctttggtcgttcccccatccctgcccccctgagcacttcttttactccccccaccgcccccgctctttctctgtctctgtttttttcttttccagaaaatcagttcgaggaaagggaaaggggcaaaaacgaaagcgcaagaaatctagatataagtcctggagcgtgtaagttggtgcccgctgctgtctaatgccctggagcctccctggcccccagtacaacctccgcctgccattccctgtaaccctgcctccctcccctggtccctccctggctctcatcctcctggcccgtgtctctctctcactctctcactccactaattggcaccaacgggtagatttggtggtggcattgctggtccagggttggggtgaatgggggtgccgacttggcctggaggattaagggaggggaccctggcttggctgggcaccgattttctctcacccactgggcactggtggcaggcccatgttggcacaggtgcctgctcacccaactggtttccattgctctaggcttctgcactcgtctggaagctgagggtggtggggagggcagacatggcccaagaagggctgtgaatgactggaggcagcttgctgaatgactccttggctgaaggaggagcttgggtgggatcagacaccatgtggcggcctcccttcatctggtggaagtgccctggctcctcacggaggtggggcctctggaggggagccccctattctggcccaacccatggcacccacagaggcctccttgcagggcagcctcttcctccgggtcggaggctgtggtgggccctgccctgggccctctggccaccagcggcctggcctggggacactgcctccgggcttagcctcccatcacaccctactttagcccaccttggtggaagggcctggacatgagccttgcacggggagaaggtggcccctgattgccatccccagcaggtgaagagtcaaggcgtgctccgatgggggcaacagcagttgggtccctgtggcctgagactcacccttgtctcccagagacacagcattgccccttatggcagcctctccctgcactctctgcccgtctgtgcccgcctcttcctgcggcaggtgtcctagccagtgctgcctctttccgccgctctctctgtcttttgctgtagcgctcggatccttccagggcctgggggctgaccggctgggtgggggtgcagctgcggacatgttagggggtgttgcatggtgattttttttctctctctctgctgatgctctagcttagatgtctttccttttgcctttttgcagtccctgtgggccttgctcagagcggagaaagcatttgtttgtacaagatccgcagacgtgtaaatgttcctgcaaaaacacagactcgagatgcaaggcgaggcagcttgagttaaacgaacgtacttgcaggttggttcccagagggcaagcaagtcagagaggggcatcacacagagatggggagagagagagagaaagagagtgagcgagcgagcgagcgggagagcgcctgagaggggccagctgcttgctcagtttctagctgcctgcctgagatctgcgaagggcgaattccagcacactggcggccgttactagtggatctgcccactctcttccccacaccagcccctagagactgaactgaaaaccctcctcagcagggagcctcttctgattaacttcatccagctctggtcacccatcagctcttaaaatgtcaagtggggactgttctttggtatccgttcatttgttgctttgtaaagtgttcccatgtccttgtcttgtctcaagtagattgcaagctcaggagggtagactgggagcccctgagtggagctgctgctcaggccggggctccctgagggcagggctggggctgttctcatactggggctttctgccccaggaccacaccttcctgtcccctctgctcttatggtgccggaggctgcagtgacccaggggcccccaggaatggggaggccgcctgcctcatcgccaggcctcctcacttggccctaaccccagcctttgttttccatttccctcagatgtgacaagccgaggcggtgaaagcttctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg